(19) United States
(12) Patent Application Publication
(10) Pub. No.: US 2021/0205329 A1
(43) Pub. Date: Jul. 8, 2021

(54) METHODS OF INHIBITING PROINFLAMMATORY NEUROIMMUNE SIGNALING AND TREATING INFLAMMATORY DISORDERS

(71) Applicants: The University of North Carolina at Chapel Hill, Chapel Hill, NC (US); University of Maryland, Baltimore, Baltimore, MD (US)

(72) Inventors: A. Leslie Morrow, Chapel Hill, NC (US); Laure Aurelian, Redwood, CA (US); Irina Balan, Lutherville, MD (US)

(21) Appl. No.: 17/056,845

(22) PCT Filed: May 20, 2019
(86) PCT No.: PCT/US2019/033053

§ 371 Date: Nov. 19, 2020

Related U.S. Application Data
(60) Provisional application No. 62/674,379, filed on May 21, 2018.

(51) Int. Cl. A61K 31/57; A61K 45/06

(52) U.S. Cl. CPC A61K 31/57; A61K 45/06

(57) ABSTRACT

Methods of inhibiting proinflammatory neuroimmune signaling as is related to the treatment of inflammatory disorders are provided. These methods include the inhibiting of toll-like receptor signaling and/or the enhancement of anti-inflammatory signaling, and in one example, the inhibiting of TLR2, TLR4 or TLR7 signaling as well as the enhancement of fracktalkine or IL-10 signaling either alone or together.

### CROSS-REFERENCE TO RELATED APPLICATIONS

This application claims benefit of U.S. Provisional Application No. 62/674,379, filed May 21, 2018, which is hereby incorporated herein by reference in its entirety.

### STATEMENT REGARDING FEDERALLY SPONSORED RESEARCH OR DEVELOPMENT

This invention was made with Government Support under Grant Nos. AA024095 and AA021261 awarded by the National institutes of Health. The Government has certain rights in the invention.

### BACKGROUND

Neurosteroids are endogenous steroids synthesized in the brain that influence neuronal and behavioral activity. First recognized in 1941 (Selye H (1941). Proc Soc Exp Biol Med 46: 116-121), various neurosteroids were found to alter CNS activity. Later studies showed that endogenous steroids (3α,5α)3-hydroxypregnan-20-one (3α,5α-THP, allopregnanolone) and (3α,5α)3,21-dihydroxypregnan-20-one (3α,5α-THDOC, tetrahydrodeoxycorticosterone), lack genomic activity at nuclear glucocorticoid or progesterone receptors (McEwen B S (1991). Trends Pharmacol Sci 12(4): 141-147), but are potent positive modulators of GABA_{A} receptors (Majewska M D, et al. (1986). Science 232: 1004-1007; Morrow A L, et al. (1987). Eur J Pharmacol 142: 483-485). They act upon synaptic and extrasynaptic GABA_{A} receptors, mediating both phasic and tonic inhibition (Harrison N L, et al. (1987). J Pharmacol Exp Ther 241: 346-353; Stell B M, et al. (2003). Proc Natl Acad Sci USA 100(24): 14439-14444). Consistent with their GABAergic activity, these steroids have anesthetic, anticonvulsant, sedative, and anxiolytic effects (Paul S M, et al. (1992). Neuroactive steroids. FASEB Journals: 2311-2322), and modulate the hypothalamic pituitary adrenal axis to reduce stress activation (Owens M J, et al. (1992). Brain Res 573: 353-355; Patchev V K, et al. (1994). Neuroscience 62: 265-271). More recent evidence shows that 3α,5α-THP has protective activity in animal models of alcoholism (Beattie M C, et al. (2017). Addict Biol 22(2): 318-330; Cook J B, et al. (2014). J Neurosci 34(17): 5824-5834), traumatic brain injury (He et al, 2004b), multiple sclerosis (Noorbakhsh F, et al. (2014). Front Cell Neurosci 8: 134; Schumacher M, et al. (2007). Pharmacol Ther 116(1): 77-106), and Alzheimer's disease (Irwin R W, et al. (2014). Prog Neurohiol. 113:40-55). Significantly, pregnenolone, progesterone and/or 3α,5α-THP also have efficacy in clinical studies of traumatic brain injury (Wright D W, et al. (2007). Ann Emerg Med 49(4): 391-402), schizophrenia (Marx C E, et al. (2007). Biol Psychiatry 61: 13S), cocaine craving (Fox H C, et al. (2013). Psychoneuroendocrinology 38(9): 1532-1544; Milivojevic V, et al. (2016). Psychoneuroendocrinology 65: 44-53), and post-partum depression (Kanes S, et al. (2017), Lancet 390(10093): 480-489). However, the mechanism of these actions is unknown.

### SUMMARY

As disclosed herein, neurosteroids inhibit proinflammatory signaling and enhance anti-inflammatory through TLR receptors independent of their activity at GABA_{A} receptors. As a consequence, neurosteroids can be used to treat many more conditions than originally believed. Moreover, compositions and methods for determining when a neurosteroid will be effective are also provided, in some embodiments, these effects are mediated through TLR4. In some embodiments, these effects are further mediated through TLR2 and TLR7. In some embodiments, these effects are mediated through the induction of the anti-inflammatory chemokine fracktalkine (CX3CL1).

Therefore, disclosed herein is a method for treating a TLR-mediated inflammatory condition in a subject that involves administering to the subject a neurosteroid, wherein the inflammatory condition has its origins inside or outside of the central nervous system, and may be non-responsive to GABAergic drugs.

In some embodiments, the neurosteroid is pregnenolone or (3α,5α)3-hydroxypregnan-20-one (3α,5α-THP) or a combination of both steroids. The neurosteroid may also be an analog of these steroids that shares the ability to inhibit TLR signaling and/or enhance fracktalkine signaling. In some embodiments, the neurosteroid is an inhibitor of toll-like receptor signaling or corticotropin (CRF) releasing hormone signaling. In some embodiments, the neurosteroid is an inhibitor of TLR4 receptor signaling, TLR2 signaling, TLR7 signaling, or any combination thereof.

In some embodiments, the TLR-mediated inflammatory condition is a medical disorder that is non-responsive to GABAergic drugs or steroids acting at glucocorticoid receptors. In some embodiments, the TLR-mediated inflammatory condition is selected from the group consisting of sepsis, gastrointestinal disease, chronic obstructive pulmonary disease (CORD), asthma, and atherosclerosis. In some embodiments, the TLR-mediated inflammatory condition is selected from the group consisting of pain, stroke, seizure, alcohol detoxification, Alzheimer's disease, and dementia.

The disclosed method can further involve assaying a sample from the subject for TLR signaling in peripheral blood mononuclear ceils or cerebrospinal fluid, wherein decreased TLR signaling is an indication of a therapeutically effective amount of neurosteroid. The method can also further involve increasing the amount of neurosteroid administered to the subject if decreased TLR signaling in the peripheral blood mononuclear ceils or cerebrospinal fluid is not detected.

Also disclosed herein is a method for treating an inflammatory disorder in a subject in need thereof that involves detecting in a sample from the subject elevated levels of one or more of MCP-1, TNF-α, pIRF7, INF-γ or HMGB1 or deficient levels of fracktalkine or IL-10, or any combination thereof, and administering to the subject a therapeutically effective amount of a neurosteroid. In some embodiments, the method further involves monitoring samples from the subject for levels of fracktalkine, IL10, MCP-1, TNF-α, pIRF7, INF-γ and HMGB1, or any combination thereof and administering neurosteroids to attain an appropriate balance of pro-inflammatory and anti-inflammatory modulators.

In some embodiments, the inflammatory disorder is a chronic neuropsychiatric disorder. For example, the neuropsychiatric disorder can be selected from a group consisting of cognitive disorders, seizure disorders, movement disorders, traumatic brain injury, secondary psychiatric disorders, substance-induced psychiatric disorders, attentional disorders, and sleep disorders, in some embodiments, the neuropsychiatric disorder is alcoholism.

In some embodiments, the TLR-mediated inflammatory condition is a disorder that is non-responsive to GABAergic drugs. In some embodiments, the TLR-mediated inflammatory condition is selected from the group consisting of sepsis, gastrointestinal disease, chronic obstructive pulmonary disease (CORD), asthma, and atherosclerosis, in some embodiments, the TLR-mediated inflammatory condition is selected from the group consisting of pain, stroke, seizure, alcohol detoxification, Alzheimer's disease, and dementia.

Also disclosed herein is a method for identifying inhibitors of proinflammatory neuroimmune signaling that involves measuring of inhibition of MD-2 binding to TLR4 in the presence of a candidate compound, wherein the inhibition of MD-2 binding to TLR4 by a candidate compound is indicative that the candidate compound is an inhibitor of proinflammatory neuroimmune signaling.

Also disclosed herein is a method for identifying inhibitors of proinflammatory neuroimmune signaling that involves measuring of inhibition of GABA_{A} α2 subunit protein binding to TLR4 in the presence of a candidate compound, wherein the inhibition of GABA_{A} α2 subunit protein binding to TLR4 by a candidate compound is indicative that the candidate compound is an active agent for treating a neuropsychiatric disorder.

In some embodiments, the candidate compound is a neurosteroid, or a modification, variant, derivative, or analog thereof. In some embodiments, the inhibition of MD-2 binding to TLR4 is measured by immunoprecipitation. In some embodiments, the method further comprises measuring of inhibition of any one of, any number of, or all of, pTAK1, TRAF8, NFκB p50, phospho-NF-κB-p65, pCREB, HMGB1, MCP-1 and TNFα, pIRF7 or INF-γ.

Also disclosed herein is a method for identifying inhibitors of proinflammatory neuroimmune signaling in brain that involves measuring of inhibition of GABA_{A}R α2 subunit binding to TLR4 in the presence of a candidate compound, wherein the inhibition of GABA_{A}R α2 subunit binding to TLR4 by a candidate compound is indicative that the candidate compound is an inhibitor of proinflammatory neuroimmune signaling in neurons. In some embodiments, the candidate compound is a neurosteroid, or a modification, variant, derivative, or analog thereof. In some embodiments, the inhibition of GABA_{A}R α2 subunit binding to TLR4 is measured by immunoprecipitation. In some embodiments, the method further comprises measuring of inhibition of upregulation of, any number of, or all of, pTAK1, TRAF6, NFκB p50, phospho-NFkB 50, NFkB p65 phospho-NF-κB-p6S, pCREB, HMGB1, MCP-1, TNFα, pIRF7 or INF-γ.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts 3α,5α-THP inhibiting LPS-activated TLR4 signaling in RAW264.7 cells. RAW264.7 ceils were treated with IPS (1 μg/ml) and 3α,5α-THP (0.5 μM or 1 μM) and harvested after 24 hrs. The levels of pTAK1 [F₁₉=50.47, n=5/grp], MCP1 [F₁₉=97.27, n=5/grp], TRAF6 [F₁₉=26.54, n=5/grp], NF-κB p50 [F₁₉=19.89, n=5/grp], phospho-NF-κB p65 [F₁₉=37.95, n=5/grp], pCREB [F₁₉=89.06, n=5/grp], HMGB1 [F₁₉=19.64, n=5/grp], and TNF-α [F₁₅==29.62, n=4/grp] were significantly increased in LPS-treated vs. untreated cells (CTL), but the increase was inhibited with 3α,5α-THP at both doses studied (*p≤0.05, by One-way ANOVA; Newman-Keuls post-hoc test). 3α,5α-THP (0.5 μM, p=0.3385, n=5/grp or 1 μM, p=0.6947, n=5/grp) did not affect TLR4 expression.

FIG. 2 depicts pregnenolone inhibiting LPS-activated TLR4 signaling in RAW264.7 cells. RAW264.7 cells were exposed to IPS (1 μg/ml) and pregnenolone (0.5 μM or 1 μM) and harvested 24 hours later. The levels of pTAK1 [F₁₉=90.0, n=5/grp], MCP1 [F₁₉=100.56, n=5/grp], TRAF6 [F₁₉=38.96, n=5/grp], NF-κB p50 [F₁₉=19.72, n=5/grp], phospho-NFκB p65 [F₁₉=38.96, n=5/grp], pCREB [F₁₉=90.04, n=5/grp], HMGB1 [F₁₉=19.72, n=5/grp], and TNF-α [F₁₅=25.54, n=4/grp] were significantly increased in the LPS-treated as compared to untreated (CTL) cells but the Increase was inhibited with pregnenolone (Preg) at both doses studied (*p≤0.05, by One-way ANOVA; Newman-Keuls post-hoc test). Pregnenolone (0.5 μM, p=0.1763, n=5/grp or 1 μM, p=0.9570, n=5/grp) did not affect TLR4 expression.

FIGS. 3A and 3B depict neurosteroids targeting the activated TLR4 signal by inhibiting TLR4/MD-2 binding. (FIG. 3A) 3α,5α-THP and pregnenolone specifically target the activated TLR4 signal. RAW264.7 cells untreated (CTL) or treated with 3α,5α-THP (THP; 1 μM) or pregnenolone (Preg; 1 μM) were harvested after 24 hrs. The levels of pTAK1, TRAF6, and MCP1 were similar in the neurosteroid-treated and untreated ceils, indicating that the neurosteroids specifically target only the activated TLR4 signal. (FIG. 3B) Neurosteroids inhibit TLR4 signal activation in RAW264.7 ceils by blocking TLR4/MD-2 binding. RAW246.7 cells were treated with IPS (1 μg/ml) without or with 3α,5α-THP (THP; 1.0 μM) or pregnenolone (Preg; 1.0 μM) and protein extracts collected at 24 hrs post-treatment were immunoprecipitated (IP) with antibody to TLR4 or TLR2. The precipitates were immunoblotted (IB) with MD-2 antibody. Normal IgG was used as control. MD-2 co-precipitated with TLR4, but not normal IgG. The levels of MD-2 co precipitating with TLR4 were significantly reduced by 3α,5α-THP (45.4±6.9%, p<0.05) or pregnenolone (57.2±7.3%, p<0.05), but neither 3α,5α-THP nor pregnenolone had any effect on the minimal, presumably background, TLR2/MD-2 interaction. HMGB1 co-precipitated with both TLR4 and TLR2 and its levels were not altered by the neurosteroids.

FIGS. 4A-4C depict 3α,5α-THP inhibiting TLR4 signal innately activated in P rat VTA by blocking TLR4/α2 binding and TLR4/MyD88 binding. (FIG. 4A) 3α,5α-THP administration (15 mg/kg) significantly reduced MCP-1 (ELISA; Student's t(16)=2.19), TRAF6 (Student's t(16)=5.74), and CRF (Student's t(16)=3.112) levels compared to vehicle controls, with no effect on TLR4 protein expression. *p<0.05 compared to control. (FIG. 4B) TLR4 binds α2 in the P rat VTA. Protein extracts from P rat VTA were immunoprecipitated (IP) with the TLR4 or α2 antibodies or normal IgG (control) and the precipitates were reciprocally immunoblotted (IB) with α2 or TLR4 antibodies. Both α2 and TLR4 were seen in the anti-α2 and anti-TLR4 (but not normal IgG) precipitates from P rat VTA, indicative of protein-protein interaction. (FIG. 4C) 3α,5α-THP inhibits TLR4/α2 and the downstream TLR4/MyD88 binding in the P rat VTA. Protein extracts obtained from P rat VTA after 3α,5α-THP (15 mg/kg) or vehicle control administration were immunoprecipitated (IP) with antibody to TLR4. The precipitates were immunoblotted (IB) with α2 antibody. Normal IgG was used as control. α2 co-precipitated with TLR4, but not normal IgG. The levels of α2 co-precipitating with TLR4 were significantly reduced by 3α,5α-THP (82.7±9.2% reduction, p<0.001), 3α,5α-THP also inhibited the binding of TLR4 to MyD88 (43.5±5.4% inhibition, p<0.05). HMGB1 bound TLR4, but binding was not altered by 3α,5α-THP.

FIGS. 5A and 5B depict 3α,5α-THDOC effects on TLR4 signaling. FIG. 5A shows 3α,5α-THDOC enhances IPS induction of pTAK1 and TRAF6, but inhibits NF-κB and MCP-1 in RAW246.7 cells. RAW284.7 cells were treated with IPS (1 μg/mi) and 3α,5α-THDOC (0.5 μM or 1 μM) and harvested after 24 hrs. The levels of TRAPS [F₁₉=65.16, n=5/grp], pTAK1 [F₁₉=117.03, n=5/grp], NF-κB p50 [F₁₉=29.17, n=5/grp] and MCP-1 [F₁₉=65.16, n=5/grp], were significantly increased by IPS vs. untreated cells (CTL). 3α,5α-THDOC further elevated TRAF6 and pTAK1 levels while inhibiting NF-κB p50 and MCP-1 levels (*p≤0.05, One-way ANOVA; Newman-Keuls post-hoc test), 3α,5α-THDOC (0.5 μM, p=0.1909, n=5/grp or 1 μM, p=0.9807, n=5/grp) did not affect TLR4 expression. FIG. 5B shows 3α,5α-THDOC treatment (15 mg/kg) enhances TRAF6 and CRF in P rats VTA. MCP-1 levels obtained via ELISA are unchanged in 3α,5α-THDOC-treated compared to untreated animals. CRF protein levels are increased in 3α,5α-THDOC-treated P rats (Student's t(13)=2.40) compared to vehicle controls as are also the TRAF6 protein levels (Student's t(14)=2.58). *p<0.05 compared to control.

FIG. 6 depicts a schematic of activated TLR4 signaling inhibited by neurosteroids. IPS and GABA_{A}R α2, respectively activate the TLR4 signal in RAW246.7 cells and P rat VTA. Signal activation Initiates with LPS-induced TLR4/MD-2 complex formation at the cell surface in RAW246.7 cells and TLR4/GABA_{A}R α2 or TLR4/MyD88 complex formation in the P rat VTA. Complex formation is followed by the intracellular signal, one direction of which is the (MyD88)-dependent pathway that activates TRAF6 and TAK1 and results in the activation (phosphorylation) of the transcription factors NF κB and CREB. An alternate pathway activates PKA/CREB (Aurelian et al., 2016). Activated (phosphorylated) transcription factors translocate to the nucleus and initiate the production of various proinflammatory mediators, including TNFα. 3α,5α-THP inhibits both the LPS/TLR4/MD-2 and α2/TLR4 complex formation and pregnenolone (Preg) inhibits the LPS/TLR4/MD-2 complex formation and thereby, both inhibit resulting intracellular signaling. The LPS-stimulated TLR4/MD-2 interaction also initiates the ability of IPS to increase HMGB1 expression, and this is also inhibited by 3α,5α-THP and pregnenolone in RAW246.7 ceils, apparently through inhibition of the TLR4/MD-2 complex formation. Released HMGB1 can bind TLR4 or/and modulate the production of proinflammatory mediators through NF-κB-dependent or NF-κB-independent signaling pathways (dashed lines) (Park et al., 2004; Yang et al., 2010; Andersson and Tracey, 2011).

FIGS. 7A to 7C show that 3α,5α-THP inhibits the TLR2 and TLR7 signals, but not the TLR3 signal in RAW264.7 cells. FIG. 7A shows RAW264.7 ceils activated by Pam3Cys (10 μg/ml) alone or Pam3Cys together with 3α,5α-THP (1 μM) for 30 min and harvested after 24 hrs. The levels of pCREB (Student's t(16)=2.32), pERK1/2 (Student's t(18)=2.42), pATF2 (Student's t(18)=2.11), and TRAF6 (Student's t(14)=2.64) were significantly increased by Pam3Cys vs. vehicle. 3α,5α-THP completely inhibited the effect of Pam3Cys on pCREB (Student's t(16)=3.05), pERK1/2 (Student's t(18)=3.29), pATF2 (Student's t(18)=2.43) and TRAF6 (Student's t(14)=2.26). FIG. 7B shows RAW264.7 ceils treated with imiquimod (IMQ; 1 μg/ml) alone or IMQ together with 3α,5α-THP (1 μM) and harvested after 24 hrs. The level of pIRF7 was significantly higher in the IMQ-treated than untreated ceils (CTL). 3α,5α-THP completely inhibited the effect of IMG on pIRF7 (Student's t(24)=5.54). FIG. 7C shows RAW264.7 cells treated with Poly(I:C) (25 μg/ml) alone or Poly(I:C) together with 3α,5α-THP (1 μM) and harvested after 24 hrs. The level of IP-10 (Student's t(8)=2.60) was significantly higher in the Poly(I:C)-treated than untreated cells (CTL). 3α,5α-THP did not inhibit the effect of Poly(I:C) on IP-10. *p<0.05, **p<0.01, ****p<0.0001.

FIG. 8 shows 3α,5α-THP inhibits the TLR7 signal, but not the TLR3 signal in P rat NAc, Protein extracts from nucleus accumbens (NAc) collected from female P, rats treated with 3α,5α-THP (15 mg/kg, IP) or vehicle (45% w/v 2-hydroxypropyl-β-cyclodextrin, IP) were immunoblotted with antibodies to TLR7, p-IRF7, IRF3, TRAF8 and β-Actin used as gel loading control and the results are expressed as densitometric units normalized to β-Actin±SEM. 3α,5α-THP administration significantly reduced TLR7 (Student's t(16)=2.15), p-IRF7 (Student's t(16)=2.23), and TRAF6 (Student's t(16)=3.43) but not IRF3 (Student's t(16)=1.37) levels compared to vehicle controls. *p<0.05, **p<0.01 compared to control.

FIG. 9 shows sex differences in baseline MCP-1 (M>F) and p-IRF7(F>M) expression in P rat NAc, Protein extracts from NAc collected from naïve female and male P rats administered 3α,5α-THP (15 mg/kg, IP) or vehicle (45% w/v 2-hydroxypropyl-p-cyclodextrin, IP) 45 min prior to sacrifice were assayed for MCP-1 using the rat MCP-1 ELISA kit (Raybiotech—ERC-MCP-1-CL; Norcross, Ga., USA) as per manufacturer's instructions or immunoblotted with antibodies to p-IRF7 and p-actin, as a gel loading control. Two-way ANOVA revealed a significant sex difference for both MCP-1 (F (1,28)=72.27, P<0.0001) and p-IRF7 (F (1,32)=9.627, P=0.0040) levels. 3α,5α-THP administration significantly reduced MCP-1 (Two-way ANOVA: F (1,28)=21.14, P<0.0001) and p-IRF7 (Two-way ANOVA: F (1, 32)=36.89, P<0.0001) levels in both female and male P rat NAc. Tukey's multiple comparisons test following Two-way ANOVA revealed *P<0.05, **p<0.005, ****P<0.0001,

FIG. 10 shows 3α,5α-THP reduced MCP-1 levels in the VTA, amygdala, and hypothalamus of both male and female P rats. MCP-1 was measured as described in FIG. 10, Two-way ANOVA revealed no significant sex difference for MCP-1 levels in the VTA (F (1,28)=2.070, P=0.1613), the Amygdala (F (1,28)=0.02030, P=0.8877), or the Hypothalamus (F (1,28)=3.144, P=0.0871). 3α,5α-THP administration significantly reduced MCP-1 levels in both female (27%) and male (21%) P rat VTA (Two-way ANOVA: F (1,28)=14.33, P<0.0001), Amygdala [female (47%) and male (58%)] (Two-way ANOVA: F (1,28)=20.92, P<0.0001), and Hypothalamus [female (27%) and male (32%)] (Two-way ANOVA: F (1,28)=31.55, P<0.0001). Tukey's multiple comparisons test following Two-way ANOVA revealed *P<0.05, **P<0.01, ***P<0.001.

FIG. 11 shows 3α,5α-THP administration to naïve female and male P rats increased the expression of fracktalkine (CX3CL1). Rats were administered VEH or 3α,5α-THP (15 mg/kg, IP) and sacrificed after 45 min. CX3CL1 was measured by ELISA (Raybiotech—ERC-CX3CL1-CL; Norcross, Ga., USA) as per manufacturer's instructions. (Two-way ANOVA: F (1,28)=13.63, P<0.001, Tukey's multiple comparisons test *P<0.05).

FIG. 12 depicts the structures of 3α,5α-THP, pregnenolone and 3α,5α-THDOC. 3α,5α-THP and pregnenolone have distinct A ring properties, but identical C/D ring features, distinct from 3α,5α-THDOC, indicating structural specificity at rings C/D for inhibition of TLR4 binding to MD-2 and MyD88-dependent signaling in RAW246.7 cells. Structural features of 3α,5α-THP at both the A ring and C/D ring are required for inhibition of HR binding to GABA_{A}R α2 subunits in VTA.

### DETAILED DESCRIPTION

In the following detailed description, embodiments of the present invention are described in detail to enable practice of the invention. Although the invention Is described with reference to these specific embodiments, it should be appreciated that the invention can be embodied in different forms and should not be construed as limited to the embodiments set forth herein. Rather, these embodiments are provided so that this disclosure will be thorough and complete, and will fully convey the scope of the invention to those skilled in the art. All publications cited herein are incorporated by reference in their entireties for their teachings.

Unless otherwise defined, ail technical terms used herein have the same meaning as commonly understood by one of ordinary skill in the art to which this disclosure belongs.

Also as used herein, the terms “treat,” “treating” or “treatment” may refer to any type of action that imparts a modulating effect, which, for example, can be a beneficial and/or therapeutic effect, to a subject afflicted with a condition, disorder, disease or illness, including, for example, improvement in the condition of the subject (e.g., in one or more symptoms), delay in the progression of the disorder, disease or illness, delay of the onset of the disease, disorder, or illness, and/or change in clinical parameters of the condition, disorder, disease or illness, etc., as would be well known in the art.

As used herein, the terms “prevent,” “preventing” or “prevention of” (and grammatical variations thereof) may refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention, in representative embodiments, the term “prevent,” “preventing,” or “prevention of” (and grammatical variations thereof) refer to prevention and/or delay of the onset and/or progression of a metabolic disease in the subject, with or without other signs of clinical disease. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

As used herein, the terms “modulate,” “modulating” or “modulation” (and grammatical variations thereof) may refer to enhancement (e.g., an increase) or inhibition (e.g., diminished, reduced or suppressed) of the specified activity. The term “enhancement,” “enhance,” enhances,” or “enhancing” refers to an increase in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase) and/or an increase in the specified activity of at least about 5%, 10%, 25%, 35%, 40%, 50%, 60%, 75%, 80%, 90%, 95%, 97%, 98%, 99% or 100%. The term “inhibit,” “diminish,” “reduce” or “suppress” refers to a decrease in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more decrease) and/or a decrease or reduction in the specified activity of at least about 5%, 10%, 25%, 35%, 40%, 50%, 60%, 75%, 80%, 90%, 95%, 97%, 98%, 99% or 100%. In particular aspects, the inhibition or reduction results in little or essentially no detectable activity (at most, an insignificant amount, e.g., less than about 10% or about 5%).

An “effective amount” or “therapeutically effective amount” may refer to an amount of a compound or composition of this invention that is sufficient to produce a desired effect, which can be a therapeutic and/or beneficial effect. The effective amount will vary with the age, general condition of the subject, the severity of the condition being treated, the particular agent administered, during the duration of the treatment, the nature of any concurrent treatment, the pharmaceutically acceptable carrier used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. (See, for example, Remington, The Science and Practice of Pharmacy (latest edition)).

Neuroimmune signaling in the brain elevates proinflammatory cytokines, chemokines, and their associated receptors to promote CNS disease in a progressive feed-forward manner (Pavlov V A, et al. (2017). Nat Neuroses 20(2): 156-166). Proinflammatory signaling through toll-like 4 receptors (TLR4) is elevated in physiological stress (Walter T J, et al. (2017). Alcohol Clin Exp Res) and traumatic brain injury (Ahmad A, et al. (2013). PLoS One 8(3): e57208) and contributes to the aforementioned neuropsychiatric conditions, including alcohol use disorders (He J, et al. (2008). Exp Neurol 210(2): 349-358; Qin L, et al. (2008). J Neuroinflammation 5: 10), other addictions (Lacagnina M J, et al. (2017). Neuropsychopharmacology 42(1): 156-177), depression (Bhattacharya A, et al. (2016). Psychopharmacology (Berl) 233(9): 1623-1636; Dantzer R, et al. (2008). Nat Rev Neurosci 9(1): 46-56), and epilepsy (Maroso M, et al. (2011). J Intern Med 270(4): 319-326).

It is well established that inflammation in the periphery induces pro-inflammatory signaling in the brain (Crews F T, et al. (2017). Neuropharmacology 122: 56-73; Samad T A, et al. (2001). Nature 410(6827): 471-475; Thomson C A, et al. (2014). J Neuroinflammation 11: 73). The TLR4-specific ligand, lipopolysaccharide (LPS), acts on macrophage TLR4 receptors causing receptor dimerization on the ceil membrane, and a cascade of protein-protein interactions that produce proinflammatory cytokines and chemokines. LPS-activation of TLR4 signaling involves formation of a TLR4/MD-2 (myeloid differentiation factor 2) complex that is followed by intracellular signals, including the myeloid differentiation primary response 88 (MyD88)-dependent pathway that activates tumor necrosis factor receptor associated factor 6 (TRAF6), transforming growth factor (TGF)-β-activated kinase 1 (TAK1), and transcription factors NF-κB and cyclic AMP response element binding protein (CREB). Activated transcription factors translocate to the nucleus and initiate a proinflammatory response that involves the production of chemokines and various proinflammatory cytokines (Chattopadhyay S, et al. (2014). Cytokine Growth Factor Rev 25(5): 533-541; Cochet F, et al. (2017). Int J Mol Sci 18(11); Irie T, et al. (2000). FEBS Lett 467(2-3): 160-164; Kim S J, et al. (2017). BMB Rep 50(2): 55-57; Lu Y C, et al. (2008). Cytokine 42(2): 145-151).

TLR4 is also activated in neurons (Okun E, et al. (2011). Trends Neurosci 34(5): 269-281), but the mechanism is still unclear. TLR4 is innately activated in neurons from P rats selectively bred for alcohol intake, but not in alcohol-non-preferring (NP) rats (Liu J, et al. (2011). Proc Natl Acad Sci USA 108(11): 4465-4470). The signal involves the γ-aminobutyric acid A receptor (GABA_{A}R) α2 subunit and controls impulsivity and the initiation of binge alcohol drinking and is sustained by a corticotropin releasing hormone (CRF) amplification loop (Aurelian L, et al. (2016). Transl Psychiatry 6: e815; Balan I, et al. (2017). Brain Behav Immun. 69:139-153; June H L, et al. (2015). Neuropsychopharmacology 40(6): 1549-1559). CRF is also known to promote TLR4 signaling (June H L, et al. (2015). Neuropsychopharmacology 40(6): 1549-1559; Tsatsanis C, et al. (2006). J Immunol 176(3): 1869-1877; Whitman B A, et al. (2013). Alcohol Clin Exp Res 37(12): 2086-2097). Both stress and alcohol induce CRF signaling and both stress and alcohol play a significant role in addiction (Dedic N, et al. (2017). Curr Mol Pharmacol. 11 (1):4-31; Gondre-Lewis M C, et al. (2016). Stress 19(2): 235-247; Koob G F, et al. (2014). Neuropharmacology 76 Pt B: 370-382; Lowery-Gionta E G, et al. (2012). J Neurosci 32(10): 3405-3413; Phillips T J, et al. (2015). Genes Brain Behav 14(1): 98-135), as well as other neuropsychiatric diseases.

To examine the possibility that 3α,5α-THP inhibits proinflammatory neuroimmune signaling in the periphery and the brain, the effects of 3α,5α-THP and pregnenolone on LPS-induced TLR4 activation was studied in mouse monocyte/macrophage RAW264.7 cells and the VTA of naïve P rats, which are established model systems for analysis of TLR4 receptor activation, as described above. Focus was on the ventral tegmental area (VTA) because both TLR4 and neuroactive steroid modulation in the VTA alter drinking behavior (Cook et al, 2014; June et al, 2015). Pregnenolone was tested because it reduces ethanol intake in P rats (Besheer et al, 2010), and shares the same steroid ring D structure of 3α,5α-THP, but lacks intrinsic potent GABAergic activity (Harrison et al, 1987; Purdy et al, 1990). 3α,5α-THP also inhibits CRF-mediated activation of the hypothalamic pituitary adrenal axis (Owens et al, 1992; Patchev et al, 1996b), but effects on extra-hypothalamic CRF are unknown.

The endogenous neurosteroid (3α,5α)3-hydroxypregnan-20-one (3α,5α-THP, allopregnanolone or brexanolone) has protective activity in animal models of alcoholism, depression, traumatic brain injury, schizophrenia, multiple sclerosis, and Alzheimer's disease that has not been well understood. Because these conditions involve proinflammatory signaling through toil-like receptors (TLRs), the effects of 3α,5α-THP and pregnenolone on LPS-induced TLR4 activation was examined in both the periphery and the CNS. Monocytes/macrophages (RAW264.7) were used as a model of peripheral immune signaling and studied innately activated TLR4 in the VTA of selectively bred alcohol-preferring (P) rats. LPS activated the TLR4 pathway in RAW284.7 ceils as evidenced by increased levels of pTAK1, TRAF8, NFκB p50, phospho-NF-κBp65, pCREB, HMGB1, and inflammatory mediators, including MCP-1 and TNFα, Both 3α,5α-THP and pregnenolone (0.5-1.0 μM) substantially (˜80%) inhibited these effects, indicating pronounced inhibition of TLR4 signaling. The levels of MD-2 co-precipitated with TLR4 were significantly reduced in the presence of 3α,5α-THP, indicating that the mechanism of inhibition of TLR4 signaling involves blockade of TLR4/MD-2 protein interactions in RAW246.7 ceils, in VTA, 3α,5α-THP (15 mg/kg, IP) administration reduced TRAF6 (˜20%), CRF (˜30%), and MCP-1 (˜20%) levels, as well as TLR4 binding to GABA_{A} α2 subunits (˜80%) and MyD88 (˜40%). These data indicate that inhibition of proinflammatory neuroimmune signaling underlies protective effects of 3α,5α-THP in immune cells and brain, by way of blocking protein-protein interactions that initiate TLR4-dependent signaling, inhibition of pro-inflammatory TLR4 signaling represents a new mechanism of 3α,5α-THP action in the periphery and the brain.

Therefore, disclosed herein is a method for administering to a subject in need thereof a compound or pharmaceutical composition for the treatment of a disorder or disorders related to proinflammatory neuroimmune signaling. For administration, either the compound or pharmaceutical composition is understood as being the active ingredient and capable of administration to a subject, and thus, in some instances, the terms are interchangeable. In some embodiments, the compounds or pharmaceutical compositions may include at least one neurosteroid. In some embodiments, the neurosteroid may be (3α,5α)3-hydroxypregnan-20-one (3α,5α-THP, allopregnanolone). In some embodiments, the neurosteroid may be pregnenolone. In some embodiments, the neurosteroid may be ganaxolone. In other embodiments, the compounds or pharmaceutical composition may include more than one neurosteroid, in some embodiments, the neurosteroid may be a therapeutically effective modification, variant, derivative, or analog of 3α,5α-THP or pregnenolone. In some embodiments, the compound or pharmaceutical composition may include the following compound: (3α,5α)3-hydroxypregnan-20-one (3α,5α-THP, allopregnanolone)

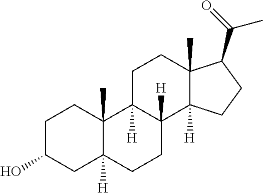

or a modification, variant, derivative, or analog thereof.

Subjects suitable to be treated using the methods of the present invention include, but are not limited to mammalian subjects. Mammals according to the present invention include, but are not limited to, canines, felines, bovines, caprines, equines, ovines, porcines, rodents (e.g., rats and mice), lagomorphs, primates, humans and the like, and mammals in utero. Any mammalian subject in need of being treated or desiring treatment according to the present invention is suitable. Human subjects of any gender (for example, male, female or transgender) and at any stage of development (i.e., neonate, infant, juvenile, adolescent, adult, elderly) may be treated according to the present invention, in particular embodiments, the subject may be afflicted with, suffering from or at risk for an inflammatory disorder or condition as described in greater detail below. In some embodiments, the inflammatory disorder may be a neuropsychiatric disorder or condition; it may be alcoholism, pain resulting from a traumatic injury, brain injury, multiple sclerosis (MS) or Alzheimer's disease.

The method of administration of compounds or pharmaceutical compositions is not particularly limited, and any method that would be appreciated by one of skill in the art for the compounds or pharmaceutical compositions in a particular formulation as described herein.

Compounds or pharmaceutical compositions of the present invention are suitable for oral, rectal, topical, inhalation (e.g., via an aerosol) buccal (e.g., sub-lingual), vaginal, topical (i.e., both skin and mucosal surfaces, including airway surfaces), transdermal administration and parenteral (e.g., subcutaneous, intramuscular, intradermal, intraarticular, intrapleural, intraperitoneal, intrathecal, intracerebral, intracranially, intraarterial, or intravenous), although the most suitable route in any given case will depend on the nature and severity of the condition being treated and on the nature of the particular active agent which is being used. Further, in preparing such pharmaceutical compositions comprising the active ingredient or ingredients in admixture with components necessary for the formulation of the compositions, other conventional pharmacologically acceptable additives may be incorporated, for example, carriers, excipients, stabilizers, antiseptics, wetting agents, emulsifying agents, lubricants, sweetening agents, coloring agents, flavoring agents, isotonicity agents, buffering agents, antioxidants and the like. As the additives, there may be mentioned, for example, starch, sucrose, fructose, dextrose, lactose, glucose, mannitol, sorbitol, dermabase, precipitated calcium carbonate, crystalline cellulose, carboxymethylcellulose, dextrin, gelatin, acacia, EDTA, magnesium stearate, talc, hydroxypropylmethylcellulose, 2-hydroxypropyl-β-cyclodextrin, sodium metabisulfite, and the like.

In further embodiments, the present invention provides kits including one or more containers comprising pharmaceutical dosage units comprising an effective amount of one or more compounds used in carrying out the present invention.

In some embodiments, the disorder or disorders related to proinflammatory neuroimmune signaling to be treated by the methods of the invention may be a neuropsychiatric disorder or condition. Neuropsychiatric disorders may, with no particular limitation, include: addictions, such as substance abuse, gambling, food, sex and alcoholism; childhood and development disorders, such as attention deficit hyperactivity disorder (ADHD), autism, fetal alcohol syndrome and tic disorders; eating disorders, such as anorexia nervosa and bulimia nervosa; degenerative diseases, such as dementia, Parkinson's disease and Alzheimer's disease; mood disorders, such as bipolar disorder, depression and mania; neurotic disorders, such as obsessive compulsive disorder (OCD), trichotillomania and anxiety disorder; psychoses, such as, but not limited to, hallucinations, delusions, bizarre behaviors, difficulty assimilating with society and social expectations, and disorganized thinking, which may include, but is not limited to schizophrenia; and sleep disorders, such as sleep apnea, narcolepsy, insomnia, parainsomnia and REM. In some embodiments, the disorder or disorders related to proinflammatory neuroimmune signaling may be alcoholism. In other embodiments, the disorder or disorders may be a result of traumatic injury (including, but not limited to brain), in still other embodiments, the disorder or disorders may be multiple sclerosis (MS), in still other embodiments, the disorder or disorders may be Alzheimer's disease. In an embodiment, methods of the invention are directed toward the treatment of alcoholism.

In some embodiments, the proinflammatory neuroimmune signaling related to a disorder or disorders may include signaling through toll-like receptors (TLRs). TLRs include TLR1, TLR2, TLR3, TLR4, TLR5, TLR6, TLR7, TLR8, TLR9, TLR10, TLR11, TLR12, and TLR13. In one embodiment, the proinflammatory neuroimmune signaling related to a disorder or disorders includes signaling through the toll-like receptor TLR2, TLR4 and/or TLR7. In other embodiments, the proinflammatory neuroimmune signaling related to a disorder or disorders includes signaling through any TLR that couples to MyD88 to activate proinflammatory signals.

In some embodiments, the methods of the invention are related to administration of a compound or composition in order to modulate proinflammatory neuroimmune signaling, in an embodiment, the modulation of proinflammatory neuroimmune signaling includes modulation of signaling through toil-like receptors. The modulation may include inhibition of toll-like receptor signaling, in some embodiments, the modulation may include the activation of anti-inflammatory signaling like, for example, through fracktalkine or IL-10.

The inhibition of toll-like receptor signaling may include interference with the interactions that result in the production of proinflammatory cytokines and chemokines. For example, with TLR4, lipopolysaccharide (IPS) interacting with TLR4 triggers the interaction between TLR4 and myeloid differentiation factor 2 (MD-2), which results in an increase in levels of pTAK1, TRAF6, NFκB p50, phospho-NF-κB-p65 and pCREB, and inflammatory mediators, including HMGB1, MCP-1 and TNFα. In some embodiments, the inhibition of TLR4 signaling includes inhibiting the LPS-induced upregulation of the levels of any one of, any number of, or all of, pTAK1, TRAF6, NFκB p50, phospho-NF-κB-p65 and pCREB, and inflammatory mediators, including HMGB1, MCP-1 and TNFα. In some embodiments, the inhibition of TLR4 signaling may include the inhibition of the interaction between TLR4 and MD-2. In an embodiment, the inhibition of TLR4 signaling may include the inhibition of the upregulation of HMGB1 expression.

Other embodiments of the invention may include methods of identifying candidate compounds for inhibiting proinflammatory neuroimmune signaling, and methods for identifying candidate compounds or active agents for treating inflammatory disorders. The methods of identifying candidate compounds may include examining the effect of candidate compounds on the modulation of toll-like receptor signaling, for example, the inhibition of TLR4 signaling, and the effect of the candidate compound on LPS-induced activation of TLR4, for example, the inhibition of the upregulation of the levels any one of, any number of, or all of, pTAK1, TRAF6, NFκB p50, phospho-NF-κB-p65 and pCREB, and inflammatory mediators, including HMGB1, MCP-1 and TNFα. In some embodiments, the methods of identifying candidate compounds may include examining the effect of the candidate compound on the interaction between TLR4 and MD-2, for example, the inhibition of the interaction between TLR4 and MD-2. The methods of identifying candidate compounds through the modulation of any of the interaction and/or activation of upregulation may be determined according to any method as would be appreciated by one of skill in the art.

### EXAMPLE EMBODIMENTS

1. A method for inhibiting proinflammatory neuroimmune signaling comprising the administration of an effective amount of a neurosteroid.

2. The method of embodiment 1, wherein the neurosteroid is pregnenolone or (3α,5α)3-hydroxypregnan-20-one or a combination of both steroids.

3. The method of embodiment 1 or 2, wherein the inhibiting of proinflammatory neuroimmune signaling comprises inhibiting toll-like receptor signaling or corticotropin (CRF) releasing hormone signaling.

4. The method of embodiment 3, wherein the inhibiting of toil-like receptor signaling comprises inhibiting TLR2, TLR4, or TLR7 receptor signaling or a combination of any of these TLRs.

5. A method of inhibiting toil-like receptor signaling comprising the administration of an effective amount of a neurosteroid.

6. The method of embodiment 5, wherein the neurosteroid is pregnenolone or (3α,5α)3-hydroxypregnan-20-one.

7. The method of embodiment 5 or 6, wherein the inhibiting of toil-like receptor signaling comprises inhibiting TLR2, TLR4, or TLR7 receptor signaling or a combination of any of these TLRs.

8. The method of any one of embodiments 5-7, wherein said method further comprises inhibiting CRF signaling.

9. A method for treating an inflammatory disorder in a subject in need thereof comprising the administration of a therapeutically effective amount of a neurosteroid.

10. The method embodiment claim 9, wherein the neurosteroid is pregnenolone or (3α,5α)3-hydroxypregnan-20-one or a combination of both steroids.

11. The method of embodiment 9 or 10, wherein the treating an inflammatory disorder comprises inhibiting toll-like receptor signaling or CRF signaling.

12. The method of embodiment 11, wherein the treating comprises the inhibiting of toll-like receptor signaling.

13. The method of embodiment 11 or 12, wherein the inhibiting of toll-like receptor signaling comprises inhibiting TLR2, TLR4, or TLR7 receptor signaling or a combination of any of these TLRs.

14. The method of any one of embodiments 9-13, wherein the inflammatory disorder is a chronic neuropsychiatric disorder.

15. The method of any one of embodiments 9-14, wherein the neuropsychiatric disorder is selected from a group consisting of cognitive disorders, seizure disorders, movement disorders, traumatic brain injury, secondary psychiatric disorders, substance-induced psychiatric disorders, attentional disorders, and sleep disorders.

16. The method of any one of embodiments 9-15, wherein the neuropsychiatric disorder is alcoholism.

17. A method for identifying inhibitors of proinflammatory neuroimmune signaling comprising measuring of inhibition of MD-2 binding to TLR4 in the presence of a candidate compound, wherein the inhibition of MD-2 binding to TLR4 by a candidate compound is indicative that the candidate compound is an inhibitor of proinflammatory neuroimmune signaling.

18. The method of embodiment 17, wherein the candidate compound is a neurosteroid, or a modification, variant, derivative, or analog thereof.

19. The method of embodiment 17 or 18, wherein the inhibition of MD-2 binding to TLR4 is measured by immunoprecipitation.

20. The method of any one of embodiments 17-19, wherein the method further comprises measuring of inhibition of upregulation of any one of, any number of, or ail of, pTAK1, TRAF6, NFκB p50, phospho-NF-κB p65, pCREB, HMGB1, MCP-1, pIRF-7, INFs and TNFα.

21. The method of embodiment 19, wherein the method further comprises measuring of inhibition of upregulation of HMGB1.

22. A method of identifying an active agent for treating a neuropsychiatric disorder comprising measuring of inhibition of MD-2 binding to TLR4 in the presence of a candidate compound, wherein the inhibition of MD-2 binding to TLR4 by a candidate compound is indicative that the candidate compound is an active agent for treating a neuropsychiatric disorder.

23. The method of embodiment 22, wherein the candidate compound is a neurosteroid, or a modification, variant, derivative, or analog thereof.

24. The method of embodiment 22 or 23, wherein the inhibition of MD-2 binding to TLR4 is measured by immunoprecipitation.

25. The method of any one of embodiments 22-24, wherein the method further comprises measuring of inhibition of upregulation of any one of, any number of, or all of, pTAK1, TRAF6, NFκB p50, phospho-NFκB p6S, pCREB, HMGB1, MCP-1, pIRF7, INFs and TNFα.

26. The method of embodiment 25, wherein the method further comprises measuring of inhibition of upregulation of HMGB1.

27. The method of any one of embodiments 22-26, wherein the neuropsychiatric disorder is a chronic neuropsychiatric disorder.

28. The method of any one of embodiments 22-27, wherein the neuropsychiatric disorder is selected from a group consisting of cognitive disorders, seizure disorders, movement disorders, traumatic brain injury, secondary psychiatric disorders, substance-induced psychiatric disorders, attentional disorders, and sleep disorders.

29. The method of any one of embodiments 22-28, wherein the neuropsychiatric disorder is alcoholism.

In some embodiments, the methods of the invention may take place in vitro. In other embodiments, the methods of the invention may take place in vivo.

The present invention is more particularly described in the following examples that are intended as illustrative only since numerous modifications and variations therein will be apparent to those skilled in the art.

### EXAMPLES

### Example 1

Materials and Methods

Cells and reagents. Mouse monocyte macrophage ceils (RAW264.7) that innately express the TLR4 receptor were obtained from American Type Culture Collection (Manassas, Va., USA). The cells were grown in Dulbecco's modified Eagle's medium (DMEM) (Gibco; Gaithersburg, Md., USA) supplemented with 10% fetal bovine serum (FBS, Gemini, West Sacramento, Calif., USA), 1% penicillin/streptomycin 100× (Gibco) at 37° C. in a 5% CO₂ humidified atmosphere. The TLR4-specific ligand IPS was purchased from Sigma-Aldrich (St. Louis, Mo., USA) (Cat. #L3024) and added to the cultures (1 μg/ml) 24 hrs before cell collection.

Antibodies. The following antibodies were commercially obtained and used according to the manufacturers instructions. Rabbit anti-TRAF6 (AB_793346), mouse anti-NFκB p50 (AB_628015), mouse anti-TNFα (AB_630341), mouse anti-TLR2 (AB_628364), and mouse anti-TLR4 (AB_10611320) were from Santa Cruz Biotechnology (Santa Cruz, Calif., USA). Rabbit phospho-TAK1 (Ser412) (pTAK1) (AB_2140096), mouse phospho-NFκB p65 (Ser536) (AB_331281), rabbit phospho-CREB (Ser133) (AB_2561044) were from Ceil Signaling Technology (Danvers, Mass., USA). Mouse anti-CCL2 (MCP-1) (AB_2538512), and rabbit anti-MD-2 (AB_11155832) were from Thermo Fisher Scientific (Waltham, Mass., USA). The generation and specificity of the rabbit-derived GABA_{A}α2 antibody (W. Siegharf, Center for Brain Research, Medical University of Vienna; Vienna; Austria; AB_2532077) was previously described; it recognizes amino acids 322-357 of the α2 protein (Liu et al., 2011). Mouse anti-beta-Actin (β-Actin) (AB_2687938), and rabbit anti-HMGB1 (AB_2232989) were from Proteintech Group (Rosemont, Ill., USA), rabbit anti-MyD88 (AB_2722690) from NeoScientific (Woburn, Mass., USA), and rabbit anti-CRF (AB_2314240) from Peninsula Labs (San Carlos, Calif., USA). Horseradish peroxidase-labeled secondary antibodies were anti-rabbit IgG (AB_2099233) and anti-mouse IgG (AB_330924) from Cell Signaling Technology.

Immunoblotting. The assay used for RAW264.7 cell lysates and co-immunoprecipitation was as previously described (Aurelian et al, 2016; June et al, 2015; Liu et al, 2011). RAW246.7 cells grown on T-75 flasks (n=5 flasks/group) were lysed with radioimmunoprecipitation (RIPA) buffer [20 mM Tris-HCl (pH 7.4), 0.15 mM NaCl, 1% Nonidet P-40 (Sigma, St. Louis, Mo., USA), 0.1% SDS (sodium dodecyl sulfate), 0.5% sodium deoxycholate] supplemented with protease and phosphatase inhibitor cocktails (Sigma). The total protein was determined by the bicinchoninic acid assay (BCA, Thermo Fisher Scientific, Waltham, Mass., USA, Cat. #23228 and Cat. #1859078). The proteins (100 μg/lane) were resolved by SDS-polyacrylamide gel electrophoresis using freshly prepared 16×18 cm gels and transferred to polyvinylidene fluoride membranes (PVDF, Bio-Rad, Cat. #162-0177). Blots were blocked with 5% Blotting-Grade Blocker (Bio-Rad, Cat. #1706404; for non-phosphorylated primary antibodies) or 5% BSA (for phosphorylated primary antibodies) for 2 hrs at room temperature (RT) and exposed to primary antibody overnight (4° C.), followed by horseradish peroxidase-labeled secondary antibodies for 1 h (room temp), immunoreactive bands were visualized with the Plus-ECL kit reagents (Perkin Elmer, Waltham, Mass., USA, Cat. #NEL105001EA) followed by exposure to high-performance chemiluminescence film (Hyperfilm ECL; Amersham). Quantitation was by densitometric scanning with a Bio-Rad GS-700 imaging densitometer. Blots were stripped and re-probed with different primary antibodies 3-5 times. Each densitometric measurement was divided by the corresponding β-Actin densitometric measurement and the results [n=5/group] are expressed as the mean β-Actin-adjusted densitometric units±SEM.

Immunoblotting for whole VTA lysates was done as previously described (Carlson et al, 2013). Briefly, VTA micropunches (1 mm thick) were lysed with CelLytic MT (dialyzable mild detergent, bicine, and 150 mM NaCl; Sigma-Aldrich) and protease and phosphatase inhibitor cocktail according to the manufacturer's instructions. Total protein was determined by the BCA assay. The proteins (10 μg/lane) were resolved by NuPAGE™ 4-12% Bis-Tris Midi Protein Gel (Thermo Fisher, Waltham, Mass.) electrophoresis and transferred using the iBlot 2 Dry Blotting System (Thermo Fisher, Waltham, Mass.). Blots were then exposed to an antibody for β-actin for normalization. Proteins were detected with enhanced chemilumenescence (GE Healthcare, Amersham, UK). Membranes were exposed to film under non-saturating conditions. Densitometric analysis was conducted using NIH Image 1.57.

Co-Immunoprecipitation Assay RAW264.7 cells [treated with LPS (1 μg/ml), 3α,5α-THP (1 μM) or pregnenolone (1 μM)] were exposed to chemical protein crosslinking (Poulopoulos et al, 2009) at 24 hrs post-treatment. Briefly, the cells were incubated (20 min on ice) with 1 mM of the cleavable, membrane-permeable crosslinker DSP (Thermo Fisher Scientific, Cat. #PG82081). Rat VTA homogenates were incubated (20 min on ice) with 200 μM of DSP. The crosslinker was quenched in 1 M Tris buffer (pH 7.5) (to a final concentration of 10-20 mM), and the material was centrifuged at 21,000×g for 15 min. Proteins from the cells were extracted with Pierce IP Lysis Buffer (Thermo Fisher Scientific, Cat. #87787) supplemented with protease and phosphatase inhibitor cocktails (Sigma). Proteins from the VTA were extracted with CelLytic MT (dialyzable mild detergent, bicine and 150 mM NaCl; Sigma Aldrich, St. Louis, Mo., USA, Cat. #C3228) supplemented with protease and phosphatase inhibitor cocktails (Sigma) according to the manufacturers instructions. Co-immunoprecipitation was done as previously described [Author Publication in the Journal of Biological Chemistry], Specifically, protein lysates (250 μg) were first treated (4° C.; 30 min; on a rocker) with 0.1 μg of normal mouse IgG (EMD Millipore Corporation, San Diego, Calif., USA, Cat, #NI03) or normal rabbit IgG (Cell Signaling Technology, Danvers, Mass., USA, Cat, #2729) corresponding to the host species of the primary antibody together with 20 μl of Protein A/G Plus-Agarose beads (Santa Cruz Biotechnology, Cat. #sc-2003) and Pierce Protein A/G IgG binding buffer (up to 1 ml; Thermo Fisher Scientific, Cat, #54200), The agarose beads were removed by centrifugation (2,500 rpm; 4° C.) and the supernatants were incubated (1 h; 4° C.; on a rocker) with TLR4, α2, TLR2 antibodies or normal IgG control (5 μg/each) and Protein A/G Plus-Agarose beads (40 μl) (overnight; 4° C.; on a rocker). The immunoprecipitates were washed four times with ice-cold Pierce IP Lysis Buffer (Thermo Fisher Scientific, Cat, #87787) and the bound proteins were eluted at 95° C. (5 min) in 50 μl of denaturing solution [150 mM Tris-HCl (pH 7.0), 5.7% SDS, 14% β-mercaptoethanol, 17% sucrose, 0.04% bromthymol blue]. Proteins were resolved by SDS-polyacrylamide gel electrophoresis, transferred to PVDF membranes and immunoblotted with MD-2, HMGB1, MYD88, GABA_{A}R-α2, TLR2, or TLR4 antibodies.

3α,5α-THP Radioimmunoassay (RIA), 3α,5α-THP concentrations in the RAW264.7 cell media were measured by radioimmunoassay as described elsewhere (VanDoren et al, 2000), modified for use with cell media (Cook et al, 2014), Briefly, 3α,5α-THP was extracted from ceil media three times with 3 ml of ethyl acetate and spiked with 1000 counts per minute of [³H]3α,5α-THP for recovery. The extracts were purified using solid phase silica columns (Burdick and Jackson, Muskegon, Mich.) and used for the assay (run in duplicate) and for recovery measurement. Steroid levels in the samples were extrapolated from a concurrently run standard curve and corrected for their respective extraction efficiencies. The 3α,5α-THP antibody (1:500) was provided by the late Dr. Robert Purdy at Scripps Research Institute. Antibody specificity was previously verified and no significant cross reactivity with pregnenolone, progesterone, pregnenolone or 3α,5α-THDOC was found. The validity of the assay has been verified by gas chromatography mass spectrometry determinations (Porcu et al, 2010), 3α,5α-THP values are expressed as ng/ml of cell media.

Animals. Selectively bred, but alcohol naïve Alcohol-preferring (P) rats (male, 3-4 months old; 250-550 g) (n=7-9/group) were obtained from the Alcohol Research Center, Indiana University School of Medicine, Animals were double housed in Plexiglas cages containing corn cob bedding and food and water was available ad libitum. The colony room was maintained on a normal 12 hr light-dark cycle (light onset at 0700 hr). Procedures followed National Institutes of Health Guidelines under UNC institutional Animal Care and Use Committee approved protocols at University of North Carolina School of Medicine. Rats were habituated to handling for 7 days prior to administration of 3α,5α-THP (15 mg/kg, IP), pregnenolone (75 mg/kg, IP), 3α,5α-THDOC (15 mg/kg, IP), or vehicle (45% w/v 2-hydroxypropyl-p-cyclodextrin) and returned to their home cage. Rats were sacrificed after 45 minutes and the brain was removed and frozen at −80° C. until VTA micropunches were collected from 1 mm cryostat brain sections. This time point was selected because 3α,5α-THP is rapidly metabolized in vivo (Purdy et al, 1990), but has behavioral and pharmacological activity at this time point (Crawley et al, 1986).

ELISA. Brain tissue micropunches were lysed with Cellyte MT and the extracts were assayed for protein content by the BCA procedure (Pierce) and for MCP-1 using the rat MCP-1 ELISA kit (Raybiotech—ERC-MCP-1-CL; Norcross, Ga., USA) or for fracktalkine using the rat fractalkine ELISA kit (Raybiotech—ERC-CX3CL1-CL; Norcross, Ga., USA) as per manufacturer's instructions.

Statistics. Measures in the RAW264.7 ceils were analyzed using a one-way analysis of variance (ANOVA) followed by the multiple comparison Student-Newman-Keuls test, with p<0.05 considered statistically significant, n=5-8/group. In the VTA micropunches, values were analyzed by Student's t-test for comparison of 2 groups, with n=8/group. Analyses were performed using Graphpad Prism 5.0. Statistical details are given in the Figure Legends and Table 1.

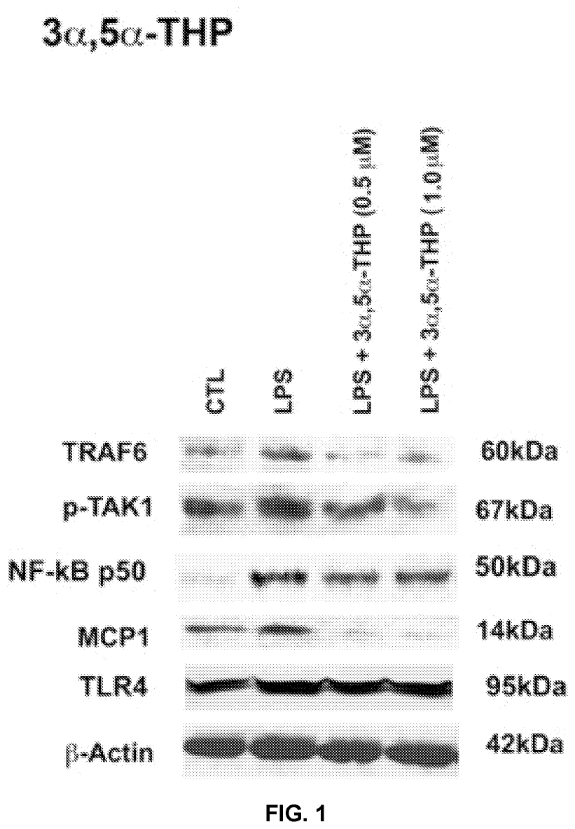

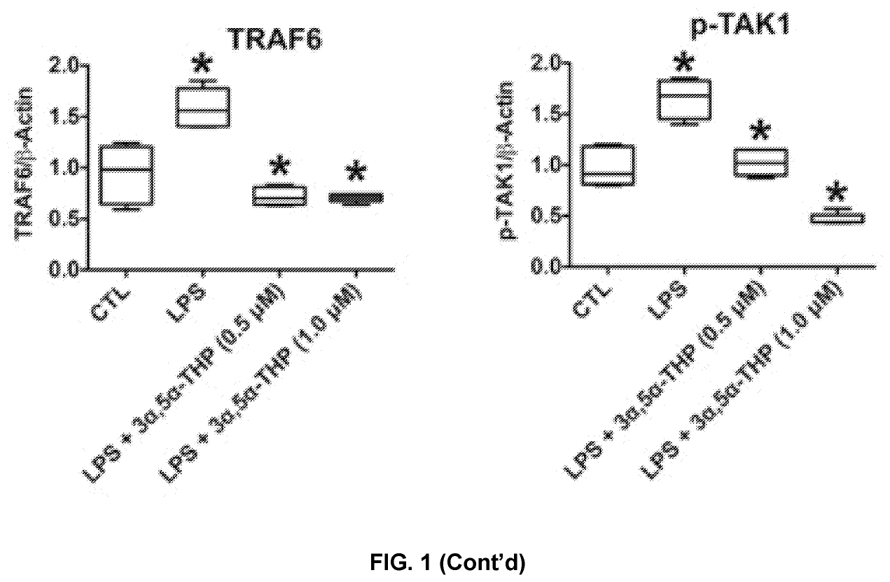

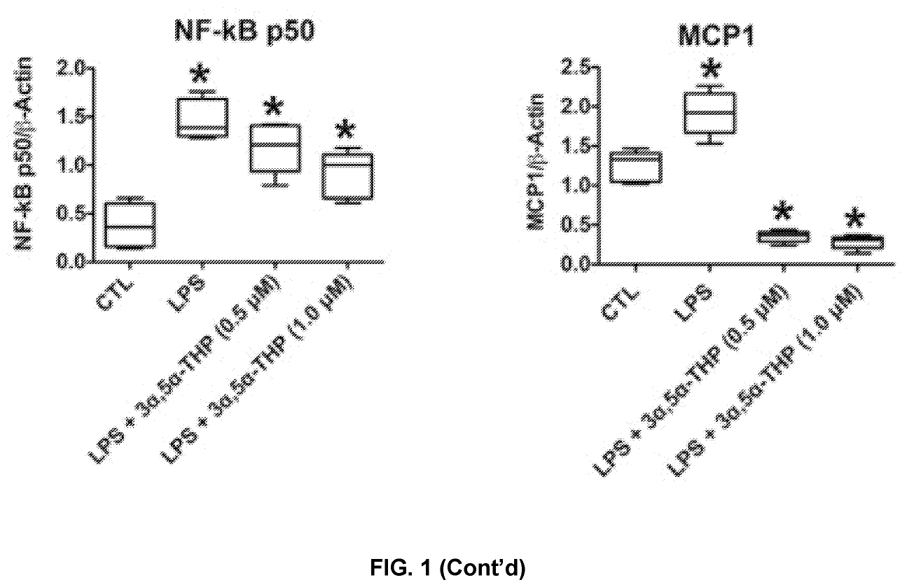

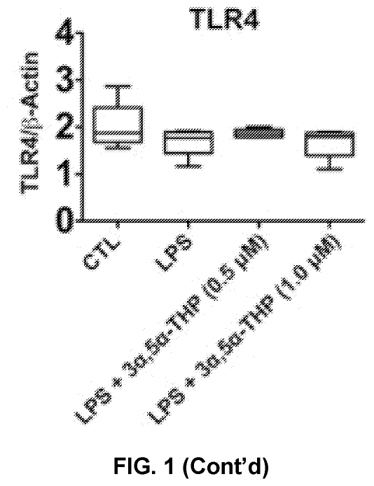

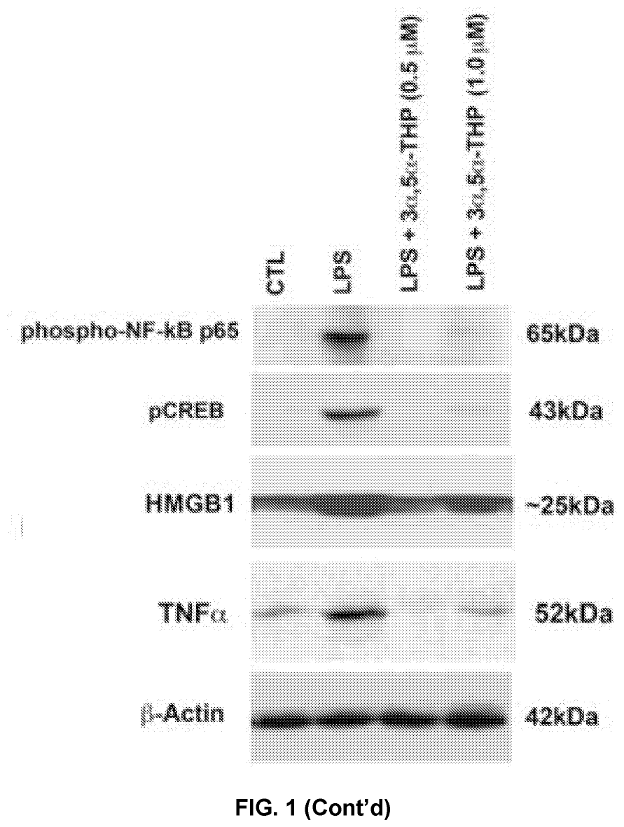

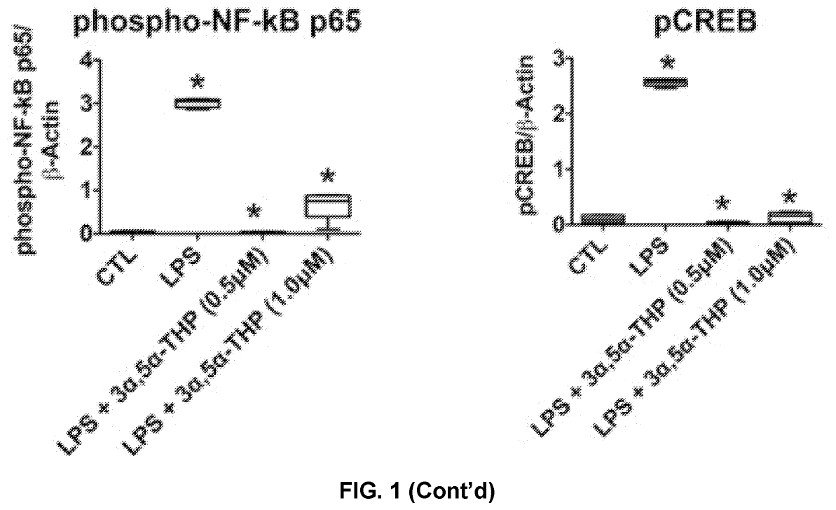

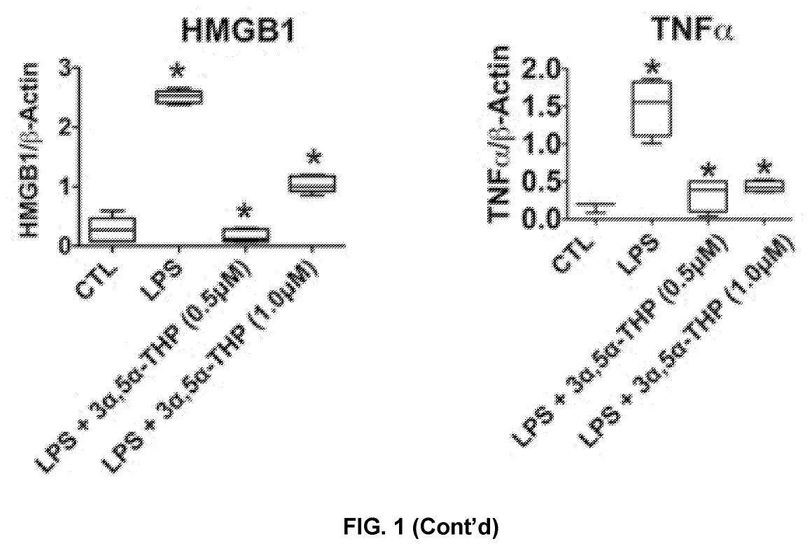

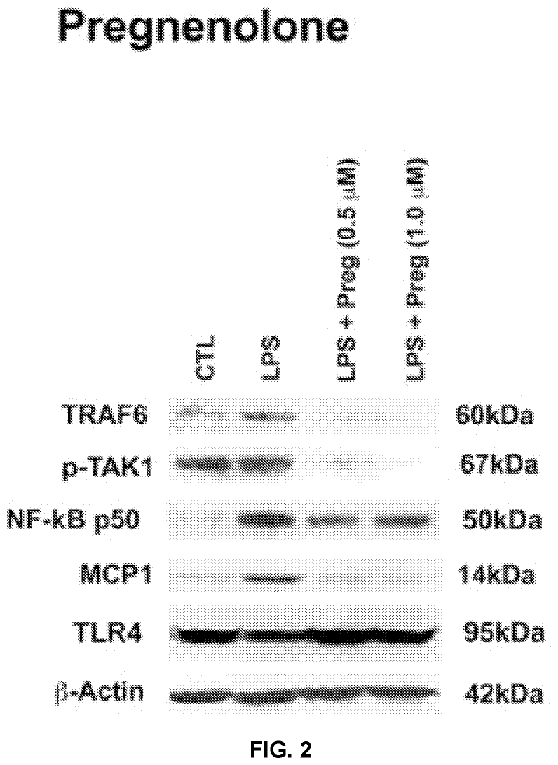

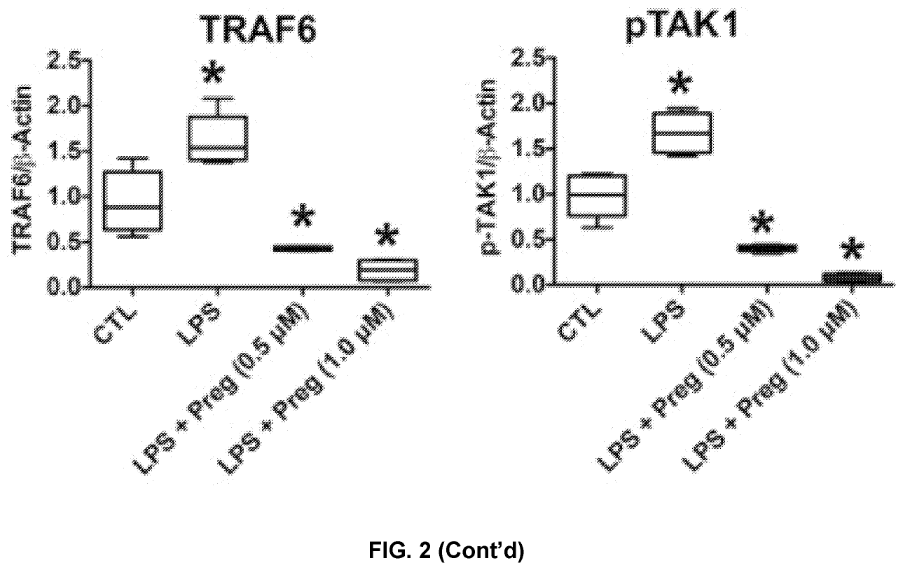

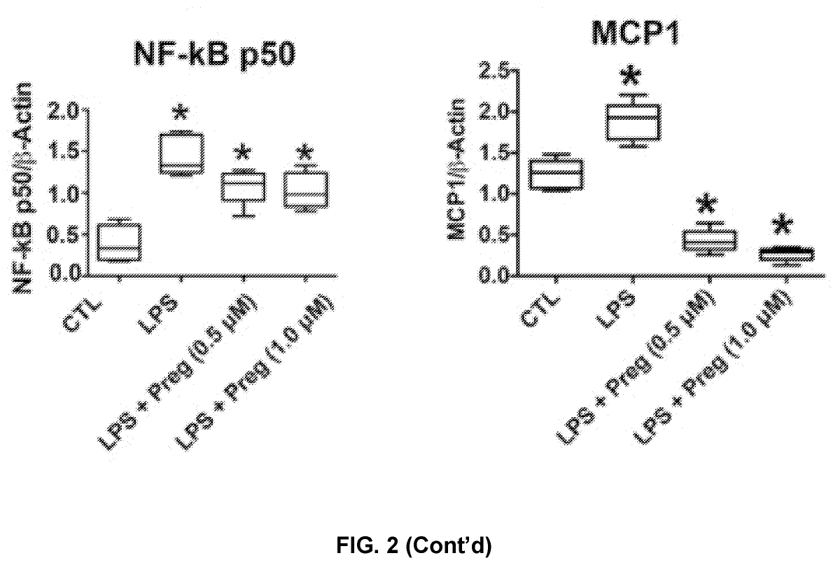

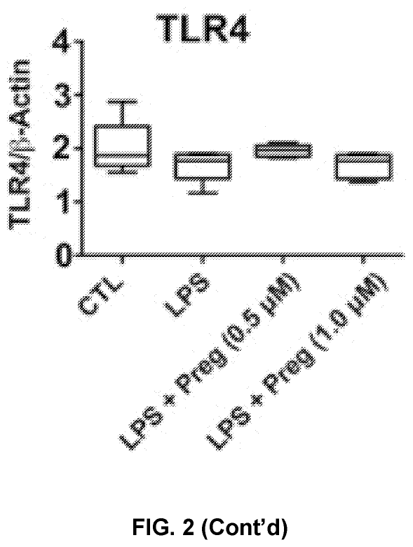

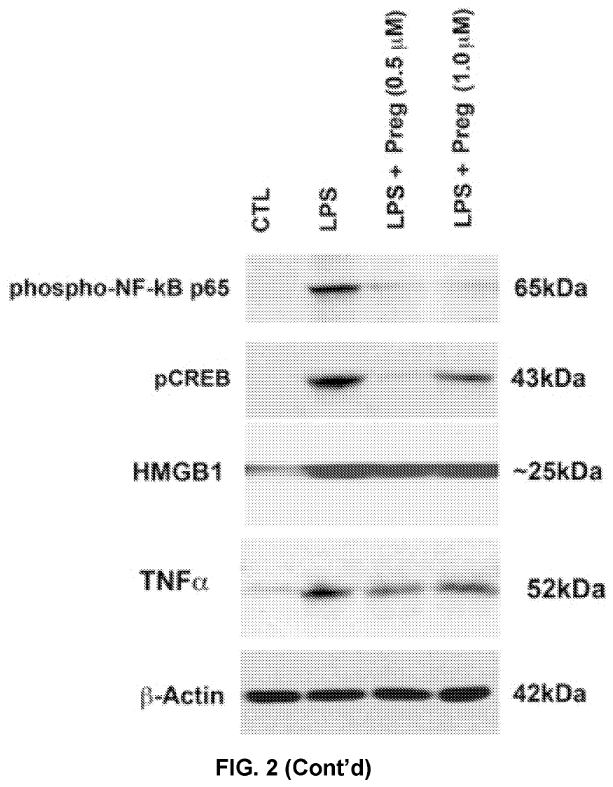

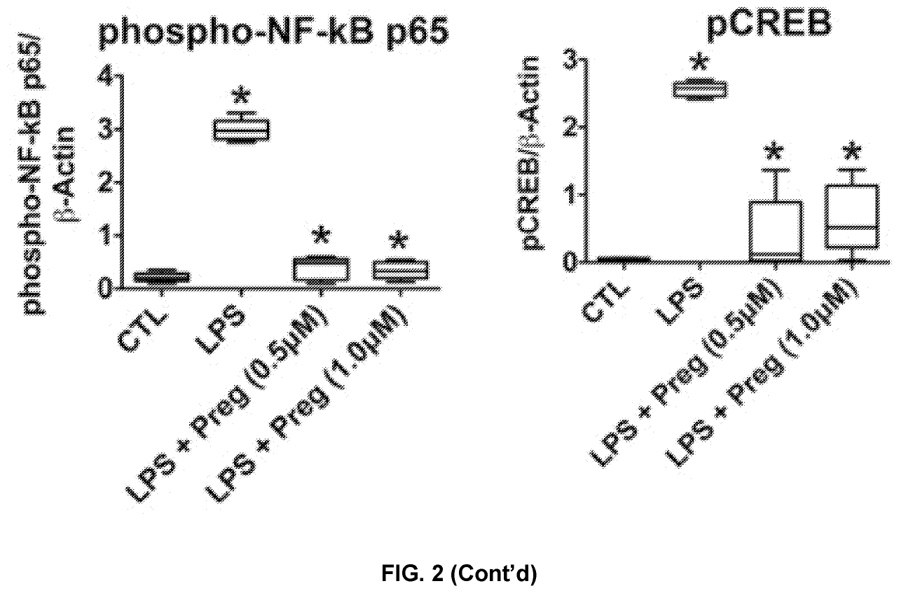

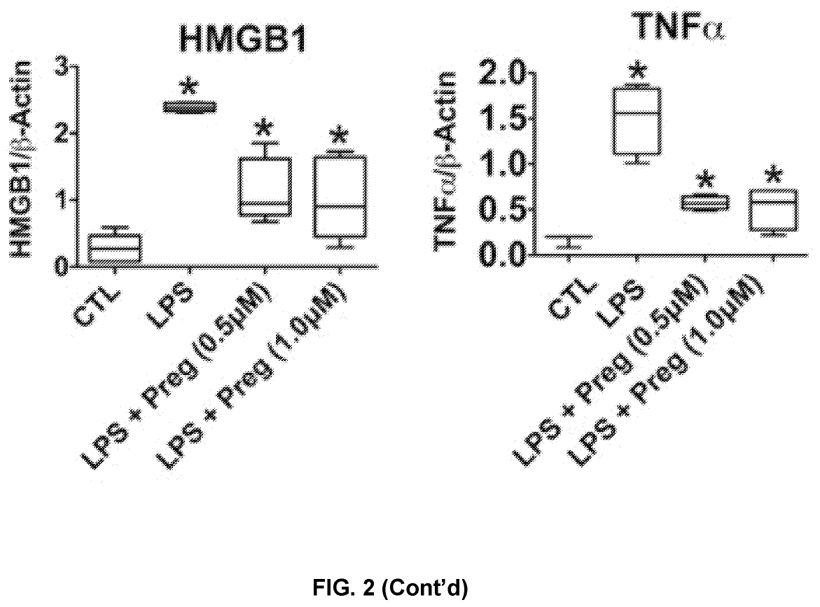

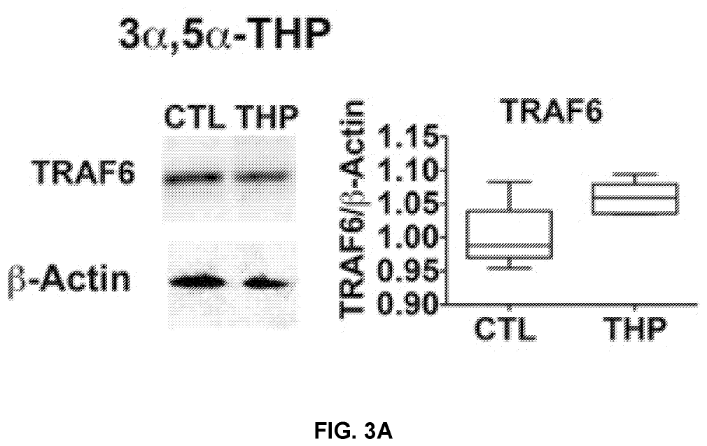

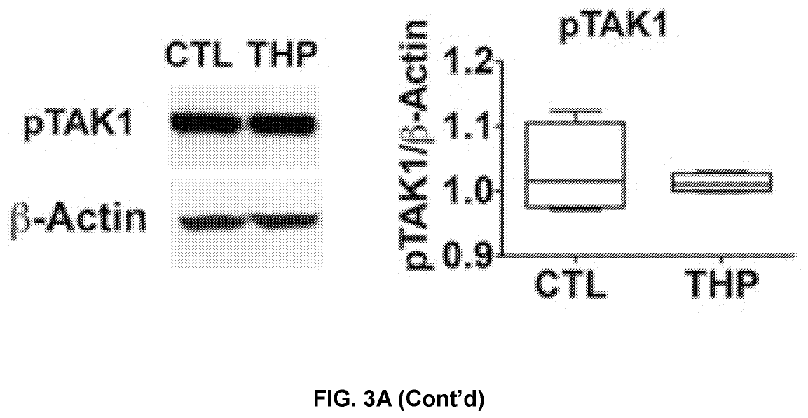

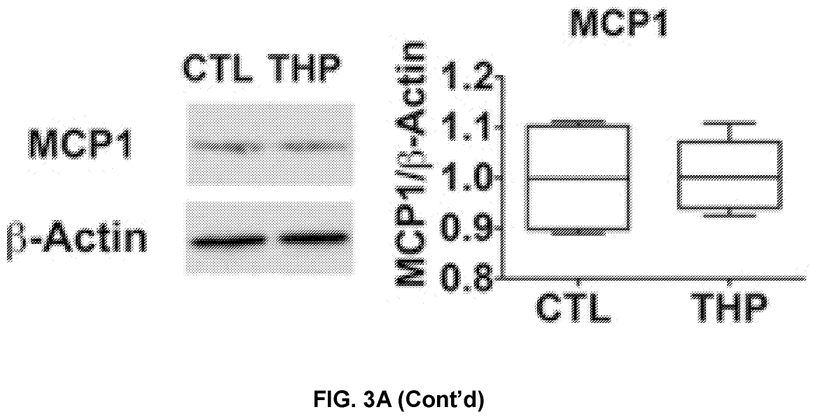

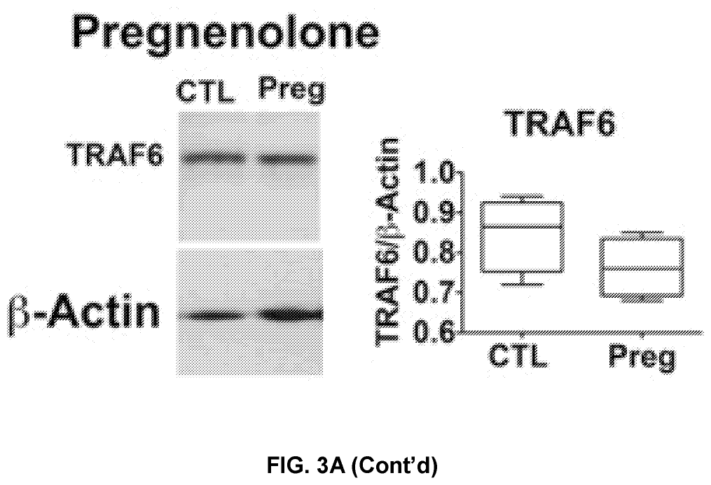

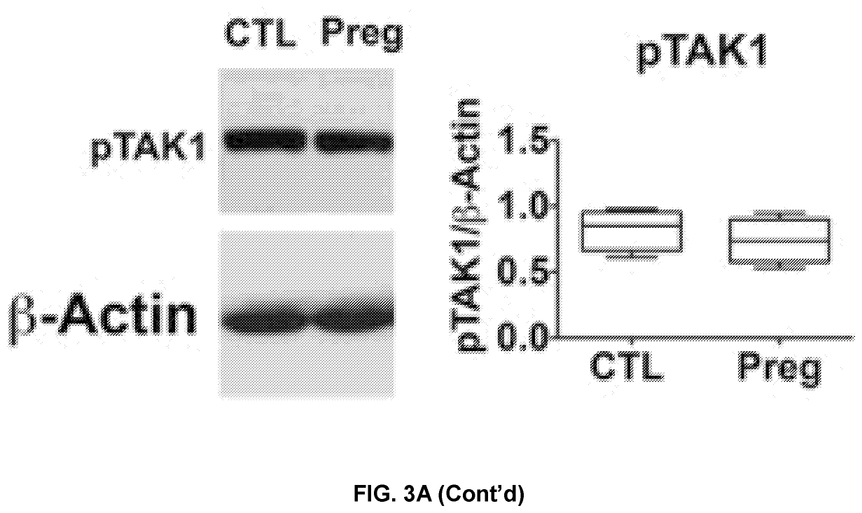

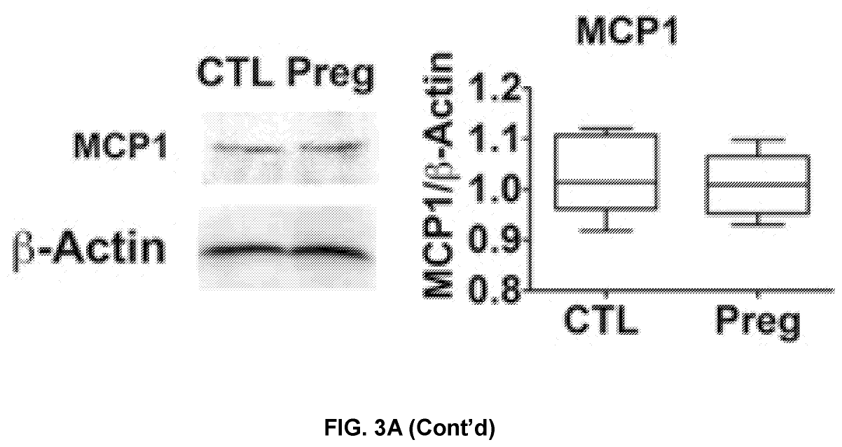

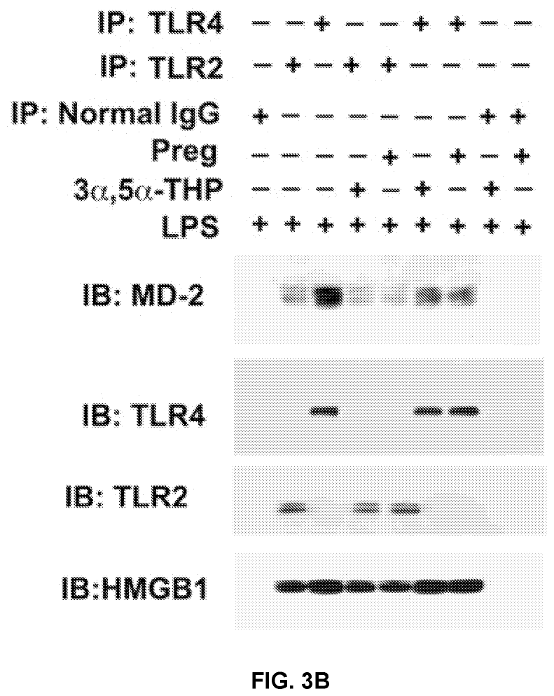

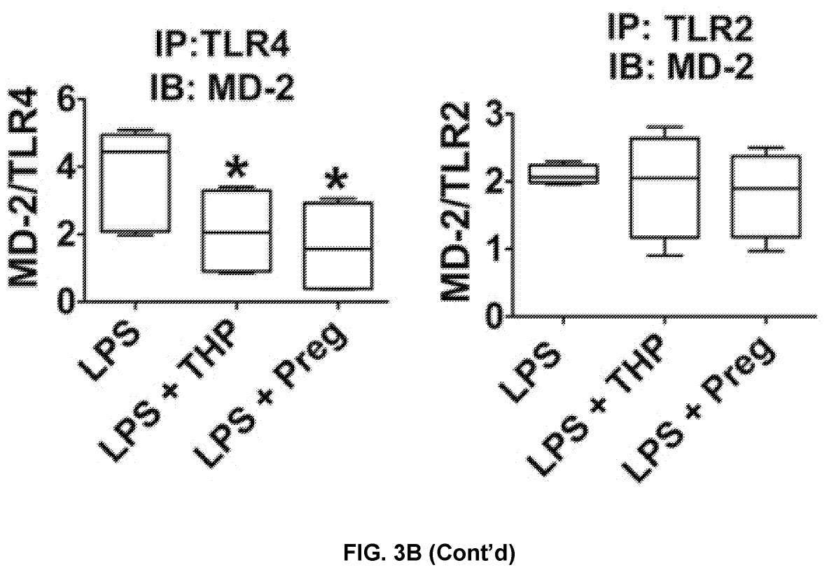

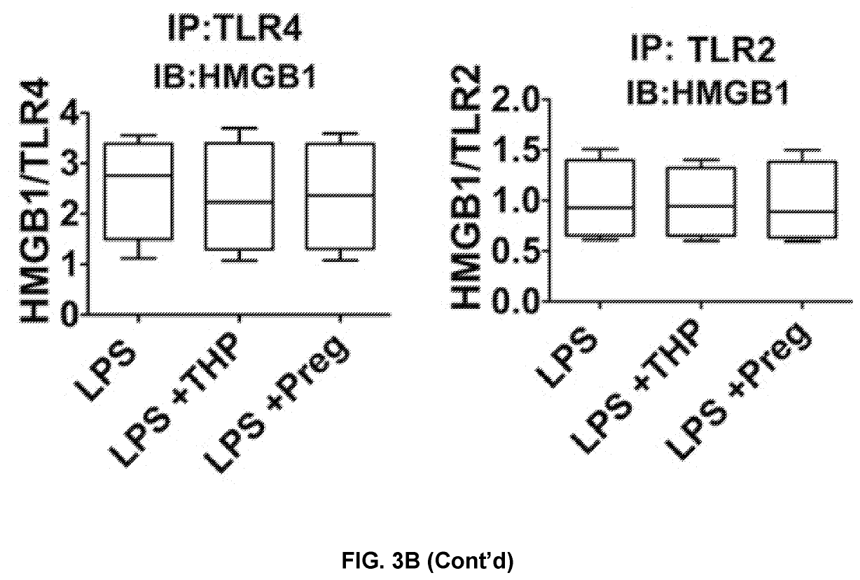

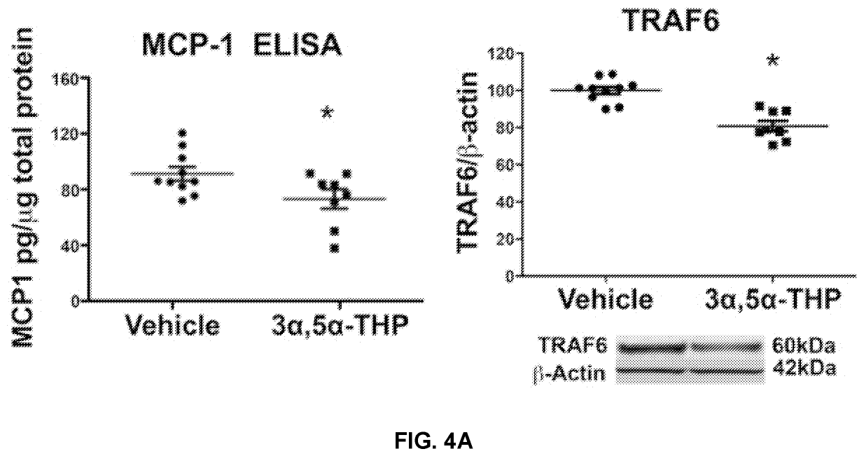

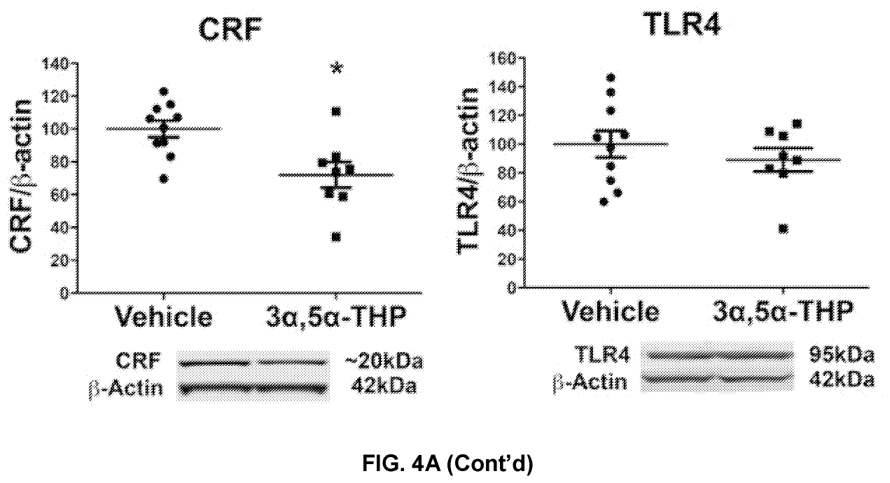

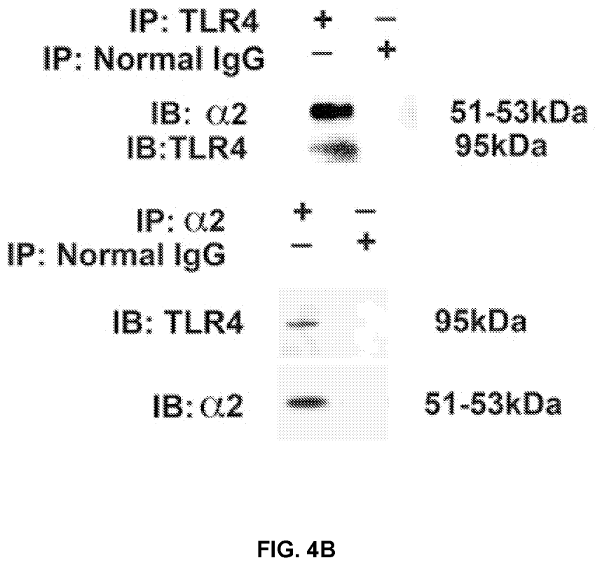

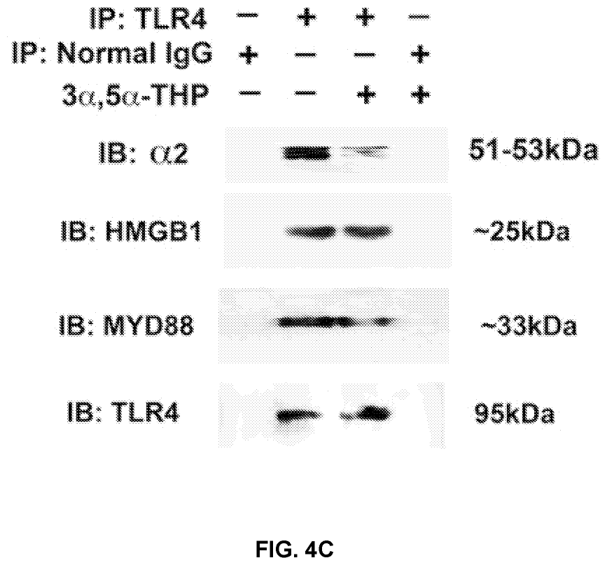

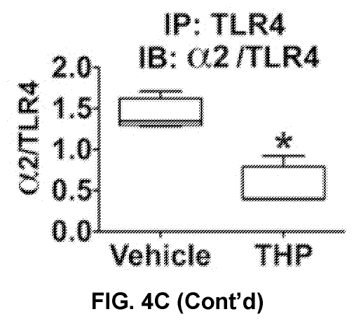

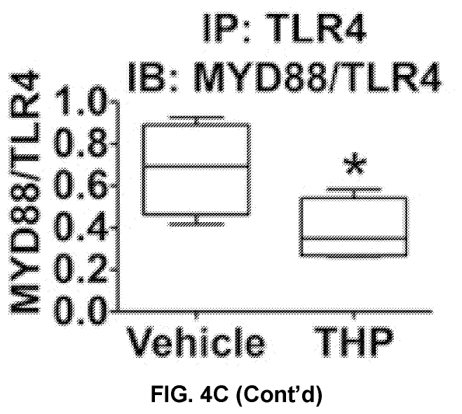

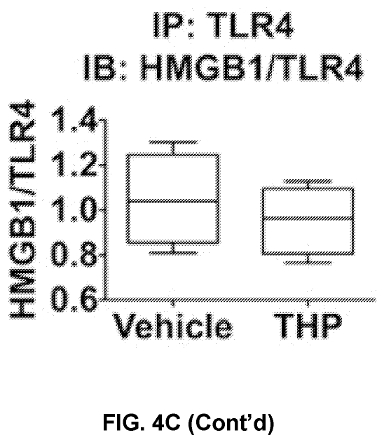

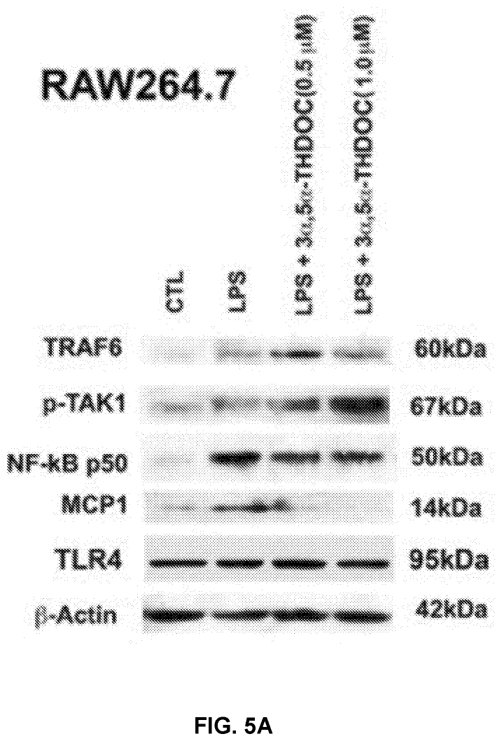

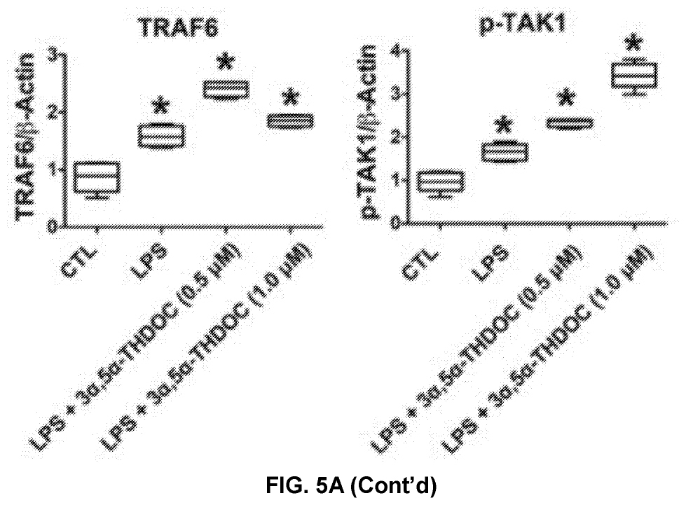

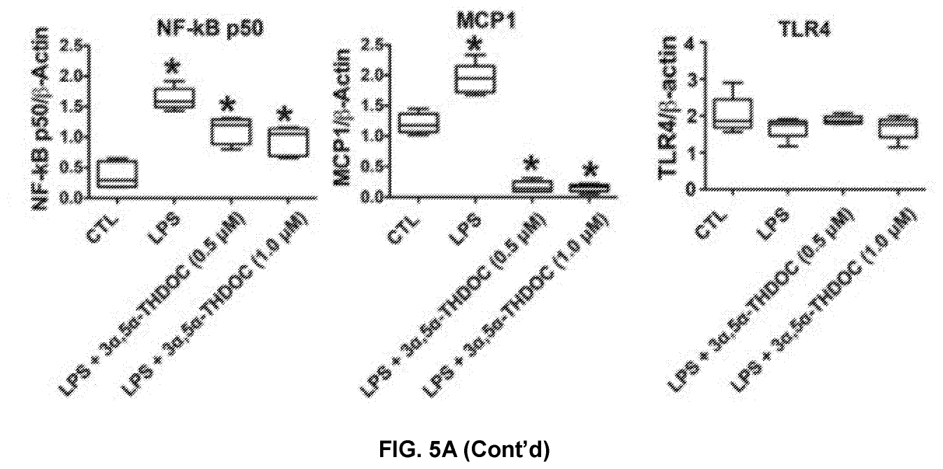

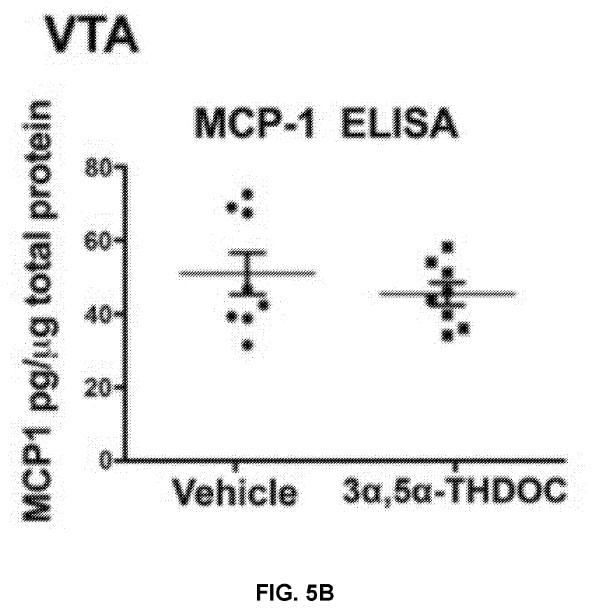

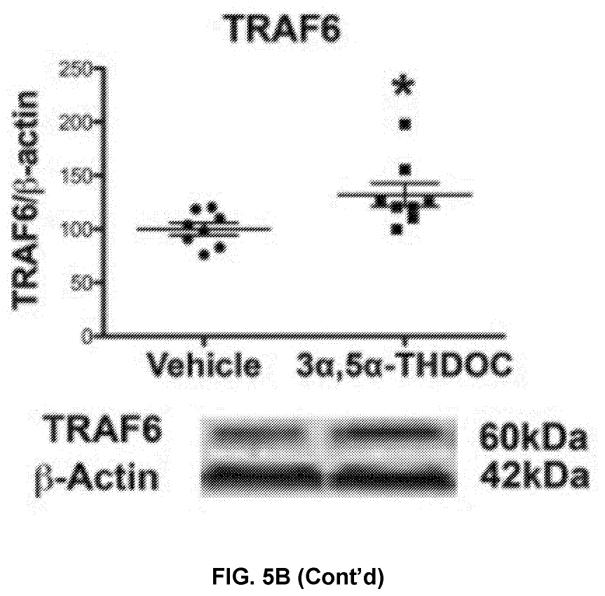

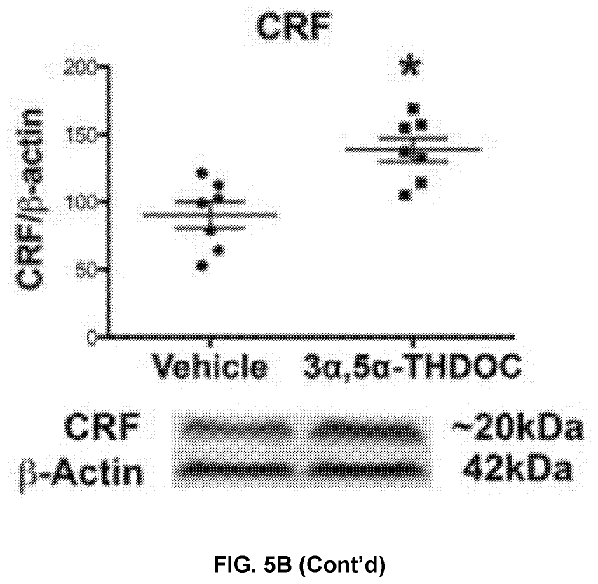

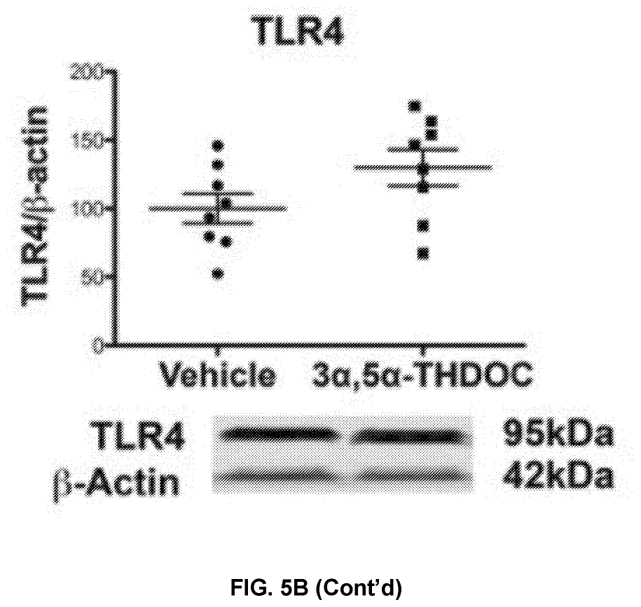

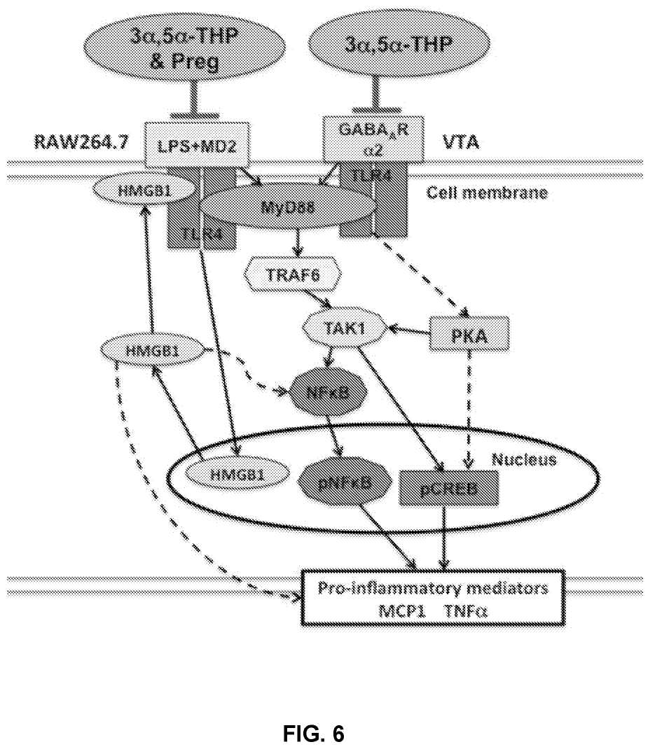

TABLE 1
Statistical Table

| Data Structure - N.D. | Statistical Test | Power |
|---|---|---|
| ^{a} FIG. 1. Effect of LPS on | One-way ANOVAs | P = 0.0000 |
| TLR signals | Newman Keuls for MCP-1 | P = 0.0193 |
| | Newman Keuls for pTAK1 | P = 0.0279 |
| | Newman Keuls for TRAF | P = 0.0034 |
| | Newman Keuls for NFkB-p50 | P = 0.0036 |
| ^{b} FIG. 2. Effect of LPS on | One-way ANOVAs | P = 0.0000 |
| TLR signals | Newman Keuls for MCP-1 | P = 0.0021 |
| | Newman Keuls for pTAK1 | P = 0.0154 |
| | Newman Keuls for TRAF | P = 0.0383 |
| | Newman Keuls for NFkB-p50 | P = 0.0044 |
| ^{c} FIG. 1. Pregnenolone | One-way ANOVA | P = 0.0000 |
| inhibition of LPS-activated | Newman Keuls for Preg 0.5 μM | P = 0.0003 |
| MCP-1 | Newman Keuls for Preg 1.0 μM | P = 0.0001 |
| ^{d} FIG. 1. Pregnenolone | One-way ANOVA | P = 0.0000 |
| inhibition of LPS-activated | Newman Keuls for Preg 0.5 μM | P = 0.0003 |
| pTAK1 | Newman Keuls for Preg 1.0 μM | P = 0.0001 |
| ^{e} FIG. 1. Pregnenolone | One-way ANOVA | P = 0.0000 |
| inhibition of LPS-activated | Newman Keuls for Preg 0.5 μM | P = 0.0006 |
| TRAF | Newman Keuls for Preg 1.0 μM | P = 0.0006 |
| ^{f} FIG. 1. Pregnenolone | One-way ANOVA | P = 0.0000 |
| inhibition of LPS-activated | Newman Keuls for Preg 0.5 μM | P = 0.0391 |
| NFkB-p50 | Newman Keuls for Preg 1.0 μM | P = 0.0161 |
| ^{g} FIG. 2. 3α,5α-THP | One-way ANOVA | P = 0.0000 |
| inhibition of LPS-activated | Newman Keuls for 3α,5α-THP 0.5 μM | P = 0.0000 |
| MCP-1 | Newman Keuls for 3α,5α-THP 1.0 μM | P = 0.0000 |
| ^{h} FIG. 2. 3α,5α-THP | One-way ANOVA | P = 0.0000 |
| inhibition of LPS-activated | Newman Keuls for 3α,5α-THP 0.5 μM | P = 0.0001 |
| pTAK1 | Newman Keuls for 3α,5α-THP 1.0 μM | P = 0.0001 |
| ⁱ FIG. 2. 3α,5α-THP | One-way ANOVA | P = 0.0000 |
| inhibition of LPS-activated | Newman Keuls for 3α,5α-THP 0.5 μM | P = 0.0001 |
| TRAF6 | Newman Keuls for 3α,5α-THP 1.0 μM | P = 0.0009 |
| ^{j} FIG. 2. 3α,5α-THP | One-way ANOVA | P = 0.0000 |
| inhibition of LPS-activated | Newman Keuls for 3α,5α-THP 0.5 μM | P = 0.0128 |
| NFkB-p50 | Newman Keuls for 3α,5α-THP 1.0 μM | P = 0.0454 |
| ^{k} FIG. 3. Effect of LPS on | One-way ANOVAs | P = 0.0000 |
| TLR signals | Newman Keuls for MCP-1 | P = 0.0114 |
| | Newman Keuls for pTAK1 | P = 0.0170 |
| | Newman Keuls for TRAF | P = 0.0047 |
| | Newman Keuls for NFkB-p50 | P = 0.0011 |
| ^{l} FIG. 3. 3α,5α-THDOC | One-way ANOVA Newman Keuls for | P = 0.0000 |
| enhancement of LPS- | 3α,5α-THDOC 0.5 μM Newman Keuls for | P = 0.0005 |
| activated TRAF | 3α,5α-THDOC 1.0 μM | P = 0.0461 |
| ^{m} FIG. 3. 3α,5α-THDOC | One-way ANOVA | P = 0.0000 |
| enhancement of LPS- | Newman Keuls for 3α,5α-THDOC 0.5 μM | P = 0.0024 |
| activated pTAK1 | Newman Keuls for 3α,5α-THDOC 1.0 μM | P = 0.0006 |
| ⁿ FIG. 3. 3α,5α-THDOC | One-way ANOVA | P = 0.0000 |
| inhibition of LPS-activated | Newman Keuls for 3α,5α-THDOC 0.5 μM | P = 0.0327 |
| NFkB-p50 | Newman Keuls for 3α,5α-THDOC 1.0 μM | P = 0.0018 |
| ^{o} FIG. 3. 3α,5α-THDOC | One-way ANOVA | P = 0.0000 |
| inhibition of LPS-activated | Newman Keuls for 3α,5α-THDOC 0.5 μM | P = 0.0002 |
| MCP-1 | Newman Keuls for 3α,5α-THDOC 1.0 μM | P = 0.0001 |
| ^{p} FIG. 4. Pregnenolone | Students t-test t(15) = 2.42 | p = 0.028 |
| effect on MCP-1 in VTA | | |
| ^{q} FIG. 5. 3α,5α-THP effect | Students t-test t(16) = 2.19 | p = 0.044 |
| on MCP-1 in VTA | | |
| ^{r} FIG. 5. 3α,5α-THP effect | Students t-test t(16) = 5.74 | p = 0.0001 |
| on TRAF6 in VTA | | |
| ^{s} FIG. 5. 3α,5α-THP effect | Students t-test t(16) = 3.112 | p = 0.007 |
| on CRF in VTA | | |
| ^{t} FIG. 6. 3α,5α-THDOC | Students t-test t(14) = 2.58 | p = 0.022 |
| effect on TRAF6 in VTA | | |
| ^{u} FIG. 6. 3α,5α-THDOC | Students t-test t(13) = 2.40 | p = 0.032 |
| effect on CRF in VTA | | |

N.D. Normal distribution

Results

3α,5α-THP and Pregnenolone Inhibit LPS-Activated TLR4 Signaling in RAW284.7 Cells

To examine whether the neurosteroids inhibit the LPS-activated TLR4 signal, RAW264.7 cells were treated with IPS (1 μg/mi; 24 hrs) in the absence or presence of 3α,5α-THP (0.5 μM, 1 μM) or pregnenolone (0.5 μM, 1 μM), and ceil extracts were assayed for expression of MyD88-dependent pathway members, by immunoblotting with antibodies to pTAK1, monocyte chemotactic protein (MCP-1), TRAF6, TLR4, and transcription factor NFκB p50 (Chattopadhyay S, et al. (2014). Cytokine Growth Factor Rev 25(5): 533-541; Irie T, et al. (2000). FEBS Lett 467(2-3): 180-164; Lu Y C, et al. (2008). Cytokine 42(2): 145-151).

The data are shown in FIGS. 1 and 2 and the statistical analysis is summarized in Table 1, where each result is indicated by alphabetical superscripts. The data show that the levels of MCP-1, pTAK1, TRAF6, and NFκB p50 were significantly increased in the LPS-treated vs. untreated cells, but these increases were blocked by 3α,5α-THP (FIG. 1) or pregnenolone (FIG. 2) at both doses. 3α,5α-THP inhibited the effect of IPS activation of TLR4 on MCP-1 by 81.5±3.8% at 0.5 μM and 85.2±4.5% at 1.0 μM (FIG. 2). Further, 3α,5α-THP inhibited the effect of LPS activation on MyD88-dependent pathway members pTAK1 by 37.8±7.7% at 0.5 μM and 71.7±3.6% at 1.0 μM and TRAF6 by 54.5±5.5% at 0.5 μM and 55.3±2.6% at 1.0 μM, and LPS-induced NFκB p50 was inhibited by 19.8±7.9% at 0.5 μM and 38.3±7.3% at 1.0 μM. 3α,5α-THP did not affect TLR4 expression (FIG. 1).

Pregnenolone inhibited the effect of LPS activation on MCP-1 by 77.3±7.3% at 0.5 μM and 85.8±4.4% at 1.0 μM. Pregnenolone inhibited the effect of LPS activation of pTAK1 by 76.2±2.0% at 0.5 μM and 95.2±2.5% at 1.0 μM. The effect of LPS activation on TRAF6 was inhibited by 73.7±1.3% at 0.5 μM and 88.5±6.8% at 1.0 μM. The effect of LPS activation on NF-κB p50 was inhibited by only 25.3±7.4% at 0.5 μM and 28.8±6.7% at 1.0 μM, indicative of the contribution of other transcription factors to the neurosteroids' effect on LPS-induced MCP-1 upregulation. Pregnenolone did not affect TLR4 expression (FIG. 2) and its effects on the TLR4-activated proteins were roughly equivalent at both doses, indicating a maximal effect was obtained at 0.5 μM.

Since pregnenolone is a precursor for 3α,5α-THP, the possibility that pregnenolone may have been converted in the RAW264.7 cells was considered by analysis of 3α,5α-THP levels in the cell culture media at the time of cell harvest, 3α,5α-THP was detected at less than 0.69±0.11 nmol/L, indicative of less than 0.1% conversion of 1.0 μM pregnenolone. This result indicates that the pregnenolone effects were not due to its conversion to 3α,5α-THP.

Pregnenolone and 3α,5α-THP Inhibit the LPS-Induced Proinflammatory Response in RAW264.7 Cells

Because the neurosteroids had relatively little effect on NFκB p50, the possibility that inhibition of other transcription factors and proinflammatory responses may be involved was considered. RAW246.7 ceils were treated as described above and protein extracts were immunoblotted with antibodies to phospho-NF-κB p65, pCREB, the proinflammatory cytokine tumor necrosis factor alpha (TNFα), and high mobility group box-1 (HMGB1), a highly conserved non-histone chromosomal protein, the translocation of which from the intra- to extra-cellular environment is a critical event in inflammatory responses. Indeed, HMGB1 is currently recognized as a cytokine secreted from activated macrophages and other inflammatory cells during the innate immune response and if is believed to function as a TLR4 ligand. HMGB1 binds to the LPS-activated TLR4/MD-2 complex, which initiates transduction of a signal that stimulates macrophage release of proinflammatory cytokines, including TNFα (Andersson and Tracey, 2011; Scaffidi et al, 2002). The data summarized in FIGS. 1 and 2 indicate that IPS caused a significant increase in the levels of phospho-NF-κB p6S and pCREB (p<0.0001), but the increase was blocked by 3α,5α-THP and pregnenolone at both 0.5 μM and 1.0 μM doses. 3α,5α-THP inhibited the effect of LPS on phospho-NF-κB p65 by 90.1±8.5%, p<0.0001 at 0.5 μM and 88.9±10.8%, p<0.0001 at 1.0 μM. 3α,5α-THP inhibited the effect of LPS on pCREB by 97.2±1.9%, p<0.0001 at 0.5 μM and 94.8±3.4%, p<0.0001 at 1.0 μM. Similar to 3α,5α-THP, pregnenolone inhibited the effect of LPS on phospho-NF-κB p65 by 86.7±7.3%, p<0.0001 at 0.5 μM and 88.1±5.5%, p<0.0001 at 1.0 μM. Pregnenolone inhibited the effect of LPS on pCREB by 84.8±9.9%, p<0.01 at 0.5 μM and 83.7±8.9%, p<0.01 at 1.0 μM. Thus, both steroids were effective in inhibiting LPS activation of nuclear transcription factors that initiate the feed-forward proinflammatory signaling.

The levels of HMGB1 (p<0.0001) and TNFα (p<0.001) were also significantly increased in the LPS-treated cells and this was inhibited by both 3α,5α-THP and pregnenolone. 3α,5α-THP inhibited the effect of IPS on HMGB1 by 88.9±11.0%, p<0.0001 at 0.5 μM and 58.6±5.5%, p<0.0001 at 1.0 μM. 3α,5α-THP inhibited the effect of LPS on TNFα by 77.8±7.3%, p<0.01 at 0.5 μM and 70.9±3.5%, p<0.01 at 1.0 μM. Similar to 3α,5α-THP, pregnenolone inhibited the effect of LPS on HMGB1 by 52.0±9.8%, p<0.01 at 0.5 μM and 57.5±12.8%, p<0.01 at 1.0 μM. Pregnenolone inhibited the effect of LPS on TNFα by 61.7±3.6%, p<0.01 at 0.5 μM and 65.1±7.7%, p<0.01 at 1.0 μM. Collectively the data indicate that the neurosteroids have a broad range of inhibitory activity in RAW246.7 cells that is centered on the activated TLR4 signaling pathways. Importantly, both 3α,5α-THP and pregnenolone (1 μM) failed to inhibit the expression of pTAK1, TRAF6, and MCP1 in non-activated RAW264.7 cells in the absence of LPS (FIG. 3A). Collectively, the data indicate the neurosteroids specifically target the activated TLR4 signal.

Neurosteroids Inhibit TLR4 Signal Activation in RAW248.1 Cells by Blocking TLR4/MD-2 Binding.

Because signaling pathways and biological function are regulated by protein-protein interaction (Chandrashekaran I R, et al. (2018). FEBS Lett 592(2): 179-189; Faraz M, et al, (2018). J Biol Chem 293(9): 3421-3435; Morita N, et al. (2017). FEBS Lett 591(12): 1732-1741), experiments were conducted to determine whether the neurosteroids interfere with the formation of the TLR4/MD-2 complex that initiates signal activation through the MyD88-dependent cascade, including TRAF6, pTAK1, and the activated transcription factors leading to the upregulation of HMGB1, MCP-1 and TNFα (Andersson U, et al. (2011). Annu Rev Immunol 29: 139-162; Yang H, et al. (2010), Proc Natl Acad Sci USA 107(26): 11942-11947). RAW246.7 cells were treated with LPS (1 μg/ml) without or with 3α,5α-THP (1.0 μM) or pregnenolone (1.0 μM) and protein extracts were collected 24 hrs post-treatment and immunoprecipitated with antibody to TLR4, Immunoprecipitation with normal IgG and antibody to TLR2 served as controls. To measure co-precipitation, the precipitates were immunoblotted with MD-2 antibody.

The data summarized in FIG. 3B, indicate that MD-2 co-precipitated with TLR4, but not normal IgG, indicative of TLR4/MD-2 binding. The levels of MD-2 co-precipitated with TLR4 were significantly reduced by treatment with 3α,5α-THP (45.4±6.9% reduction, p<0.05) or pregnenolone (57.2±7.3% reduction, p<0.05). In contrast, as a negative control, TLR2/MD-2 co-immunoprecipitation was not altered by either 3α,5α-THP or pregnenolone, indicating that both steroids inhibit TLR4/MD-2 complex formation selectively and thereby, presumably, the resulting signaling pathway. Significantly, the inhibitory effect of the neurosteroids is specific for the TLR4/MD-2 interaction that initiates the LPS-induced HMGB1 upregulation, because immunoblotting of the precipitates with HMGB1 antibody indicated that HMGB1 co-precipitates with both TLR4 and TLR2, and these protein binding interactions are not altered by the neurosteroids (FIG. 3B).

3α,5α-THP Inhibits TLR4 Signaling and TLR4 Heterodimerization in the P Rat VTA.

Since the neurosteroids inhibited the TLR4 activation signal in cultured macrophage cells, experiments were conducted to determine whether this also occurs in the brain. Selectively bred P rats that have an innately activated TLR4 signal in the VTA (Liu J, et al. (2011). Proc Natl Acad Sci USA 108(11): 4465-4470), were administered 3α,5α-THP (15 mg/kg, IP) or pregnenolone (75 mg/kg, IP), sacrificed after 45 minutes and examined for TLR4 signaling using parallel measures. Since the CRF/CRFR1 system has also been associated with alcohol drinking (Dedic N, et al. (2017). Curr Mol Pharmacol. 11(1):4-31; Koob G F, et al. (2014). Neuropharmacology 76 Pt B: 370-382; Phillips T J, et al. (2015). Genes Brain Behav 14(1): 98-135; Guadros I M, et al. (2016). Front Endocrinol (Lausanne) 7: 134), and CRF was shown to sustain the activated TLR4 signal, also in the P rat VTA (June et al, 2015), the effects of the neurosteroids on CRF were studied in parallel. 3α,5α-THP administration reduced the levels of MCP-1 by 20±9% (p<0.05), TRAF6 by 19±3% (p<0.0001), and CRF by 28±9% (p<0.01), with no effect on TLR4 protein expression (FIG. 4A). Pregnenolone administration had no effect on TRAF6, CRF, or TLR4.

Because the activated TLR4 signal is downstream of the GABA_{A}R α2 subunit in P rat brain, whether TLR4 formed a complex with the GABA_{A}R α2 subunit in P rat VTA was investigated if. Protein extracts were immunoprecipitated with antibody to TLR4, followed by immunoblotting with α2 antibody. Immunoprecipitation with normal IgG served as control. As shown in FIG. 4B, α2 co-precipitated with TLR4, but not normal IgG, and binding was confirmed by precipitation with GABA_{A}R α2 antibody and immunoblotting with TLR4 antibody. Next, co-immunoprecipitation studies were conducted in the VTA from the P rats treated with vehicle (45% w/v 2-hydroxypropyl-p-cyclodextrin) or 3α,5α-THP (15 mg/kg, IP), to determine if 3α,5α-THP alters complex formation of TLR4 with GABA_{A}R α2, MyD88 or HMGB1. FIG. 4C shows that TLR4/GABA_{A}R α2 binding in the VTA is inhibited by 3α,5α-THP (62.7±9.2%, p<0.001). Interestingly, however, TLR4/MyD88 binding was also inhibited by 3α,5α-THP (43.5±5.4%, p<0.05), indicating that 3α,5α-THP may bind TLR4 in a manner that effects its interactions with both GABA_{A}R α2 and MyD88, HMGB1 also bound TLR4, but binding was not altered by 3α,5α-THP (FIG. 4B). Collectively the data indicate that the neurosteroids inhibit the innately activated TLR4 signal in the P rat VTA, involving TLR4/α2 and TLR4/MyD88 binding. However, the precise site of protein-protein interactions and the possible contribution of proteins that serve as ligands or scaffolds to facilitate binding remain unknown.

3α,5α-THDOC has Opposing Effects on Various Components of TLR4 Signaling in RAW264.7 Cells and the VTA.

The effect of the GABAergic neurosteroid 3α,5α-THDOC on TLR4 signal activation was also measured, both in RAW264.7 cells and the P rat VTA to shed light on the structural requirements for inhibition of TLR4 signaling. 3α,5α-THDOC possesses the same A ring structure as 3α,5α-THP, but has a ring D structure that is distinct from both 3α,5α-THP and pregnenolone. In contrast to 3α,5α-THP, 3α,5α-THDOC enhanced the effect of IPS on TRAPS and pTAK1 expression, while showing inhibition of NFκB p50 and MCP-1 in macrophage RAW264.7 cells (FIG. 5A). 3α,5α-THDOC enhanced LPS-induced TRAF6 by 51.6±8.3% at 0.5 μM and 16.8±5.7% at 1.0 μM, while the effect on pTAK1 was dose dependent with a 2-fold increase at 1.0 μM. There was a simultaneous inhibition of LPS-activated NFκB p50 by approximately 30-40%^{v} and inhibition of MCP-1 by approximately 90%^{w}(FIG. 5B). Furthermore, in P rat VTA, the GABAergic steroid 3α,5α-THDOC (15 mg/kg, IP) increased both TRAF6 (32±12%, p<0.05) and CRF (39±16%, p<0.05) levels (FIG. 5B), but had no effect on MCP-1 expression. No effect on TLR4 protein was observed. These data indicate that 3α,5α-THDOC does not inhibit activated TLR4 signaling through the MyD88-dependent pathways (TRAF6 and pTAK-1) in cultured macrophages or VTA, but rather enhances TLR4 activation in both macrophages and brain suggesting a distinct interaction, possibly involving CRF, with the TLR4 signaling complex.

Discussion

These studies provide direct evidence for 3α,5α-THP and pregnenolone-mediated inhibition of TLR4 signal activation in monocyte/macrophage (RAW246.7) cell cultures and 3α,5α-THP inhibition in the VTA of alcohol-preferring P rats. Their action at the initiating protein-protein interaction event was documented, as schematically represented in FIG. 6. In RAW264.7 cells, the TLR4 agonist IPS increased the levels of pTAK1; TRAF6; transcription factors NF-κB p50, phospho-NF-κB p65, and pCREB; and the proinflammatory mediators, HMGB1, MCP-1, and TNF-α, Ail of these effects were inhibited by both neurosteroids at 0.5 and 1.0 μM doses. Neurosteroid-mediated inhibition was specific for the activated pathways and was not seen in the non-LPS treated cells. Inhibition appeared to involve the ability of 3α,5α-THP and pregnenolone to block the binding of TLR4 to MD-2, indicating that both steroids interfere with the initiating step of the LPS-mediated TLR4 signal activation step.

Pregnenolone is a precursor of 3α,5α-THP in steroidogenic ceils, but there was no evidence of the conversion of pregnenolone to 3α,5α-THP in the media of RAW264.7 cells, indicating that pregnenolone inhibition of TLR4 signaling in RAW264.7 cells is an intrinsic property of the steroid. Further, pregnenolone produced maximal effects at lower doses than 3α,5α-THP in the RAW264.7 cells, indicating that it may have greater inhibitory efficacy in the TLR4 signaling pathway. The ability of both 3α,5α-THP and pregnenolone to block the binding of TLR4 to MD-2 may be related to their identical structures in the steroid D ring.

The ability of the neurosteroids to inhibit the LPS-induced upregulation of HMGB1, apparently through inhibition of the TLR4/MD-2 complex formation is particularly interesting, as it provides novel information on the neurosteroid activity as well as the role of TLR4 in the regulation of HMGB1 expression. HMGB1 is a DNA-binding intranuclear protein, but recent studies have shown that it is an actively secreted cytokine produced by inflammatory cells during innate immune responses, placing HMGB1 at the intersection between the inflammatory responses of activated and non-activated inflammatory signals. In this context, LPS, the canonical TLR4 ligand, is recognized as an established HMGB1 inducer. However, the exact signaling pathway responsible for the LPS effect on HMGB1 and its contribution to the inflammatory response are still poorly understood. This appears to involve HMGB1 binding to TLR4/MD2 and results in the transduction of a signal that stimulates macrophage release of TNFα. The binding and signaling both require the redox-sensitive cysteine in position 106 (Yang H, et al. (2010). Proc Natl Acad Sci USA 107(26): 11942-11947) and the signaling activates the nuclear translocation of activated NF-κB (Park J S, et al. (2004). J Biol Chem 279(9): 7370-7377). However, LPS and HMGB1 signaling differ. HMGB1 binds to TLR4 with much less affinity than LPS, and it activates gene expression patterns that are distinct from the LPS-mediated expression pattern (Park J S, et al. (2004). J Biol Chem 279(9): 7370-7377; Silva E, et al. (2007). Intensive Care Med 33(10): 1829-1839; Yang H, et al. (2010). Proc Natl Acad Sci USA 107(26): 11942-11947). These data are consistent with these results in that the neurosteroids inhibit the LPS-induced TLR4/MD-2 interaction and HMGB1 upregulation. However, they do not interfere with the ability of HMGB1 to bind both TLR4 and TLR2, indicating that they regulate HMGB1 production, but not its function through TLR4 receptor binding.

In the VTA of alcohol-preferring P rats, 3α,5α-THP inhibited several components of the TLR4 signaling pathway including TRAF6 and MCP-1, as well as CRF, consistent with the data from the cultured macrophage cells. Furthermore, 3α,5α-THP inhibited TLR4 dimerization with both GABA_{A}R α2 subunit and MyD88, indicating it also blocks the TLR4 initiating steps in P rat brain, interestingly, pregnenolone did not inhibit TRAF6 or CRF, indicating that structural requirements for inhibition of TLR signaling are cell type specific, and likely related to the requirements of the binding partners—both TLR4 and GABA_{A}R α2 subunits. Inhibition of TLR4-GABA_{A}R α2 binding may require both the structure of the steroid D ring common to 3α,5α-THP and pregnenolone, as well as the A ring structure of the GABAergic neuroactive steroids (Harrison et al, 1987; Purdy et al, 1990). This hypothesis could explain the inhibitory activity of 3α,5α-THP in P rat VTA, and the lack of effect of pregnenolone. While pregnenolone lacks GABAergic activity, and failed to block TRAF6 or CRF, it may interfere with TLR4/MyD88 binding and/or the PKA—pCREB pathway in the VTA.

3α,5α-THP has potent actions at synaptic and extrasynaptic GABA_{A} receptors (Harrison M L, et al. (1987). J Pharmacol Exp Ther 241: 346-353) and inhibits stress-induced hypothalamic CRF (Owens M J, et al. (1992). Brain Res 573: 353-355; Patchev V K, et al. (1996). Neuropsychopharmacology 15: 533-540). It is apparent that GABAergic inhibition is not required for the neurosteroid effects on MyD-dependent TLR4 signaling in RAW264.7 cells or P rat VTA, as pregnenolone mimicked the effects of 3α,5α-THP and 3α,5α-THDOC failed to inhibit TRAF6 in both macrophages and VTA. Moreover, 3α,5α-THP reduced CRF in the VTA, and CRF has been shown to induce TLR4 in the VTA (June H L, et al. (2015). Neuropsychopharmacology 40(6): 1549-1559) and in macrophage ceils (Tsatsanis C, et al. (2006). J Immunol 176(3): 1869-1877).

3α,5α-THP and pregnenolone inhibition of TLR4 signaling in the periphery and 3α,5α-THP inhibition of TLR4 signaling the brain, likely contribute to the therapeutic actions of these compounds. It is well established that immune signaling via macrophages in the periphery affects brain function and may participate in the feed-forward activation of neuroimmune signaling in the brain (Crews F T, et al. (2017). Neuropharmacology 122: 56-73; Samad T A, et al. (2001). Nature 410(6827): 471-475; Thomson C A, et al. (2014). J Neuroinflammation 11: 73). Pregnenolone and 3α,5α-THP are synthesized in the adrenals, gonads, and neurons, including brain synthesis independent of peripheral precursors (Morrow A L (2007). Pharmacol Ther 116(1): 1-6). Neurosteroids, like immune factors, circulate in the bloodstream, cross the blood brain barrier and diffuse between different ceil types due to their lipophilic characteristics, exhibiting paracrine effects in many cells, so these neurosteroids may affect neuroimmune signaling at the level of macrophages, neurons, or glial ceils. However, neuroimmune signaling differs in macrophages, glial cells, and neurons (Lawrimore C J, et al. (2017). Alcohol Clin Exp Res 41(5): 939-954), consistent with the differential effects of neurosteroids in macrophages and brain.

Neuroimmune signaling through TLR receptors is activated in alcohol use disorders (Crews F T, et al. (2017). Neuropharmacology 122: 56-73; He J, et al. (2008). Exp Neurol 210(2): 349-358; Qin L, et al. (2008). J Neuroinflammation 5: 10), other addictions (Lacagnina M J, et al. (2017). Neuropsychopharmacology 42(1): 156-177), depression (Bhattacharya A, et al, (2016). Psychopharmacology (Bed) 233(9): 1623-1636; Dantzer R, et al. (2008). Nat Rev Neurosci 9(1): 46-56), epilepsy (Maroso M, et al. (2011). J intern Med 270(4): 319-326), trauma of stroke (Sayeed I, et al. (2006). Ann Emerg Med 47(4): 381-389), traumatic brain injury (Ahmad A, et al. (2013). PLoS One 8(3): e57208; He J, et al. (2004). Exp Neurol 189(2): 404-412), Alzheimer's Disease (Lehmann S M, et al. (2012). Nat Neurosci 15(6): 827-835), and multiple sclerosis (Bsibsi M, et al. (2010). J Immunol 184(12): 6929-6937). Further, 3α,5α-THP has shown efficacy against seizures (Devaud L L, et al. (1995). Alcohol Clin Exp Res 19: 350-355; Kokate T G, et al. (1996). Neuropharmacology 35: 1049-1056), alcohol reinforcement and consumption (Beattie M C, et al. (2017). Addict Biol 22(2): 318-330; Cook J B, et al. (2014), J Neurosci 34(17): 5824-5834; Morrow A L, et al. (2001). Brain Res Brain Res Rev 37: 98-109; Porcu P, et al. (2014). Psychopharmacology (Berl) 231(17): 3257-3272), cocaine craving and stress-induced craving (Fox H C, et al. (2013). Psychoneuroendocrinology 38(9): 1532-1544; Milivojevic V, et al. (2016). Psychoneuroendocrinology 65: 44-53), schizophrenia (Marx C E, et al. (2009). Neuropsychopharmacology 34(8): 1885-1903), depression (Kanes S, et al. (2017). Lancet 390(10093): 480-489), traumatic brain injury (He J, et al. (2004b). Restor Neurol Neuroses 22(1): 19-31; Wright D W, et al. (2007). Ann Emerg Med 49(4): 391-402), multiple sclerosis (Noorbakhsh F, et al. (2014). Front Cell Neuroses 8: 134), and Alzheimer's disease (Brinton R D, et al. (2006). Curr Alzheimer Res 3(1): 11-17). Our findings indicate that inhibition of TLR signaling may contribute to the therapeutic actions of neurosteroids in these conditions, all of which exhibit TLR4 activation and inflammation in the brain. Furthermore, this work may inform the development of novel neuroactive steroids under development for treatment of various neurological and psychiatric disorders to ensure efficacy comparable to or better than the endogenous steroids.

TLRs, particularly TLR4, are associated with a lifetime of alcohol consumption and adaptation, despite current disagreement about which TLRs are most important in various species (Mayfield J, et al. (2017). Neuropsychopharmacology 42(1): 376). Systemic injection of the TLR4-specific ligand IPS increases voluntary alcohol consumption in mice, and human alcoholics have elevated levels of plasma IPS (Alfonso-Loeches S, et al. (2016). Neurochem Res 41(1-2): 193-209; Blednov Y A, et al. (2011). Brain Behav Immun 25 Suppl 1: S92-S105; Crews F T, et al. (2017b). Psychopharmacology (Berl) 234(9-10): 1483-1498; Leclercq S, et al. (2012). Brain Behav Immun 26(6): 911-918; Pandey S C (2012). Br J Pharmacol 165(5): 1316-1318; Pascual M, et al. (2011). Brain Behav Immun 25 Suppl 1: S80-91). Significantly, the activated TLR4 signal also regulates impulsivity and the predisposition to initiate alcohol drinking in alcohol-naïve P rats (Aurelian L, et al. (2016). Transl Psychiatry 6: e815; June H L, et al. (2015). Neuropsychopharmacology 40(6): 1549-1559), likely indicative of the presence of an innately activated signal resulting from the selective breeding for alcohol preference. In this context, it is also important to point out that pharmacologic and genetic studies have shown that alcohol induces CRF signaling and CRF plays a significant role in addiction (Dedic N, et al. (2017). Curr Mol Pharmacol. 11 (1):4-31; Gondre-Lewis M C, et al. (2016). Stress 19(2): 235-247; Koob G F, et al. (2014). Neuropharmacology 76 Pt B: 370-382; Lowery-Gionta E G, et al, (2012). J Neurosci 32(10): 3405-3413; Phillips T J, et al. (2015). Genes Brain Behav 14(1): 98-135; Quadras I M, et al. (2016). Front Endocrinol (Lausanne) 7: 134). CRF is known to activate or enhance TLR4 signaling and it sustains the innately activated TLR4 signal in P rats (June H L, et al. (2015). Neuropsychopharmacology 40(6): 1549-1559; Tsatsanis C, et al. (2006). J Immunol 176(3): 1869-1877; Whitman B A, et al. (2013). Alcohol Clin Exp Res 37(12): 2086-2097). Thus, the data presented here may be particularly relevant for neurosteroid actions in the context of TLR activation by stress and/or alcohol addiction, conditions that are often co-morbid with depression, post-traumatic stress, and seizures.

In conclusion, inhibition of proinflammatory neuroimmune signaling can be a method for the treatment of several chronic neuropsychiatric diseases. Nonetheless, neuroimmune signaling has important protective as well as deleterious effects under various conditions and the appropriate balance is needed for optimal brain and immune function (Laing J M, et al. (2010). J Neurochem 112(3): 662-676; Sanada T, et al. (2008). J Biol Chem 283(49): 33858-33864; Vartanian K, et al. (2010). Transl Stroke Res 1(4): 252-260; Winkler Z, et al. (2017). Behav Brain Res 334: 119-128). The present data indicate a beneficial role for 3α,5α-THP in these processes. Combined with potent activity on GABA_{A} receptors and the inhibition of CRF signaling, 3α,5α-THP inhibition of proinflammatory signaling in the periphery and brain may provide a novel strategy to address inflammatory disease.

### Example 2

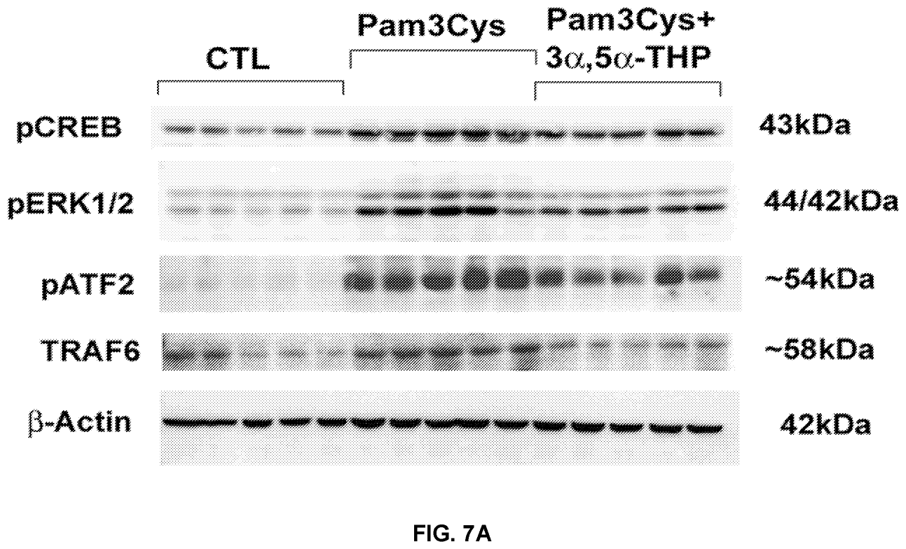

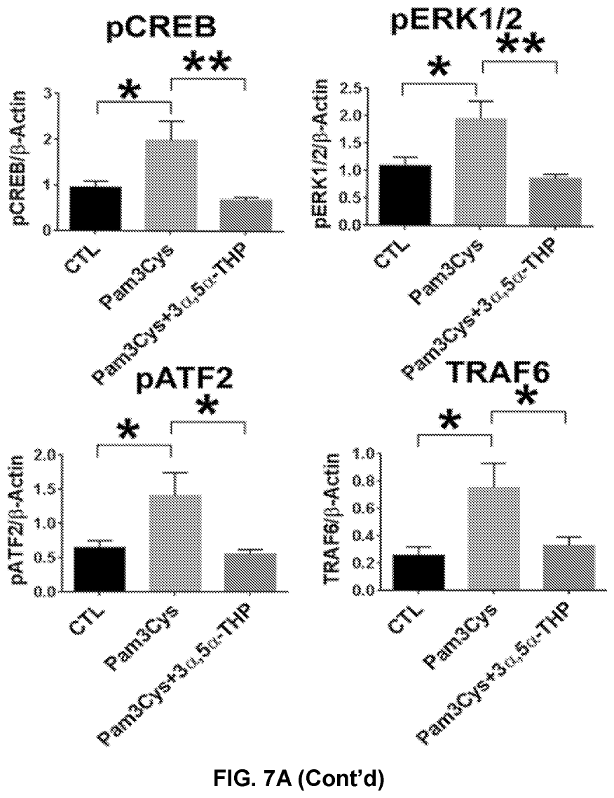

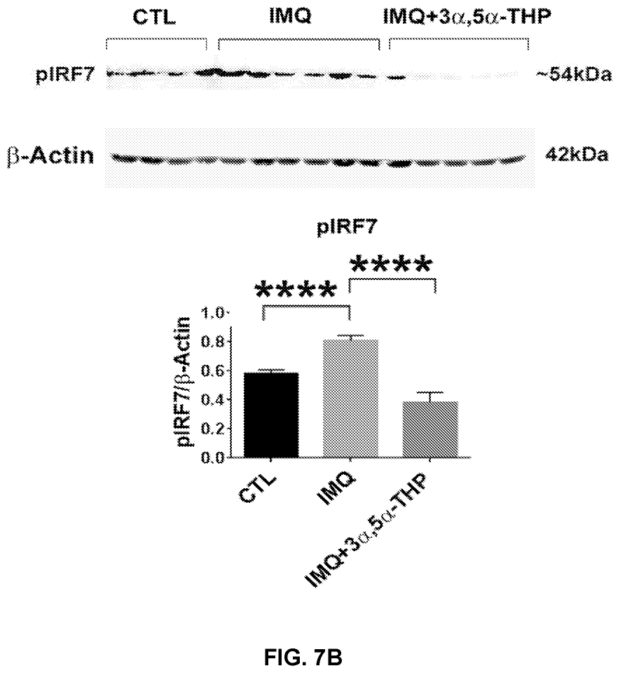

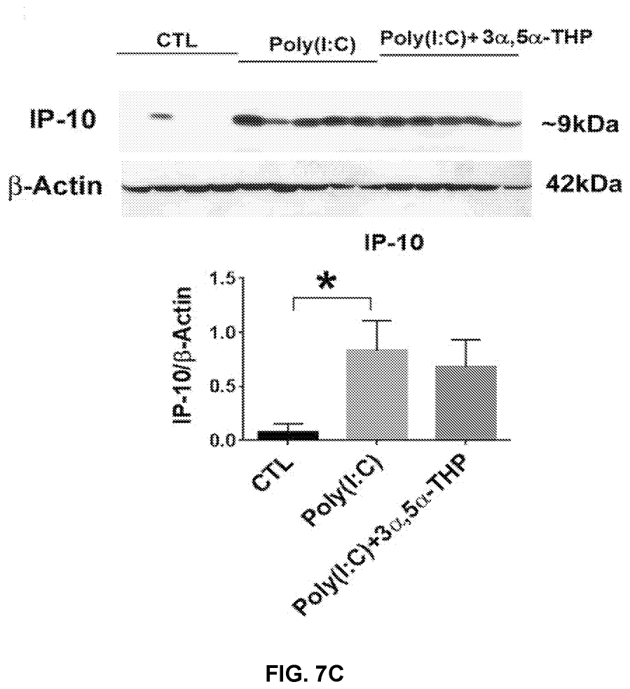

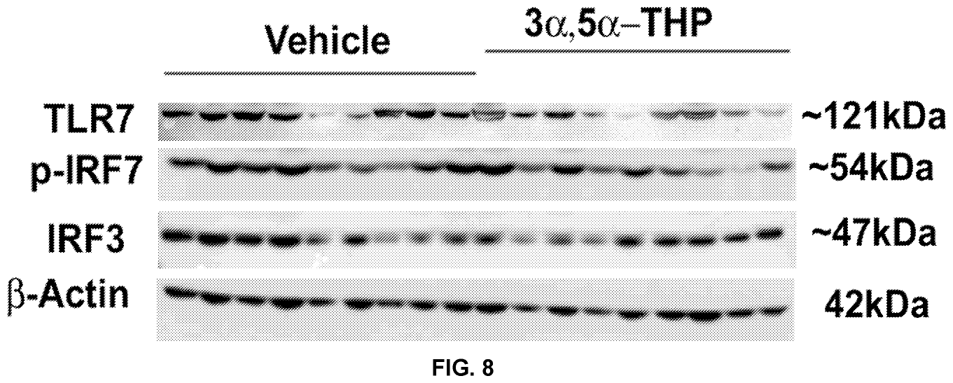

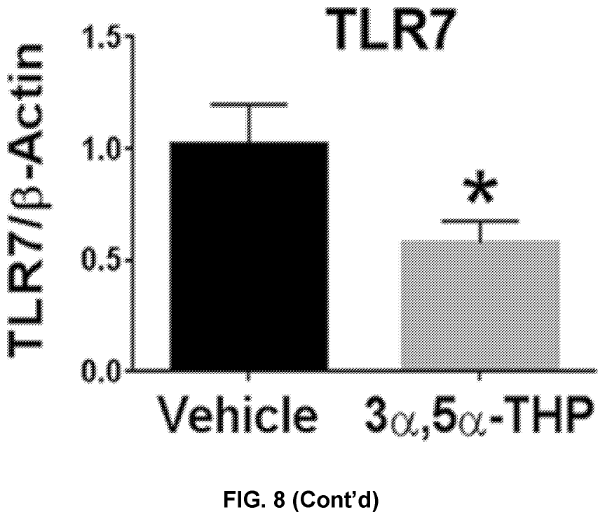

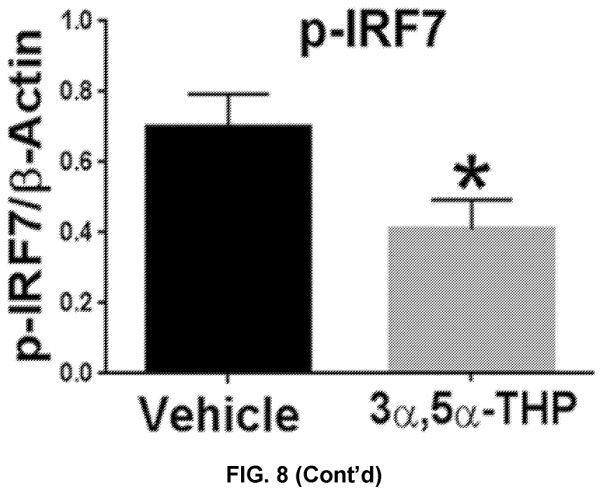

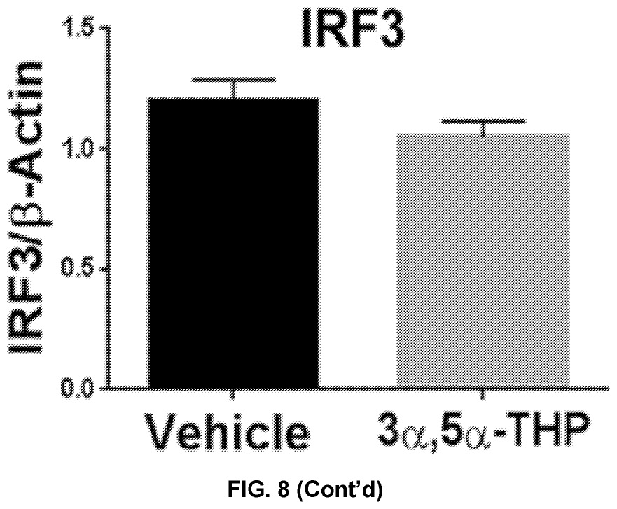

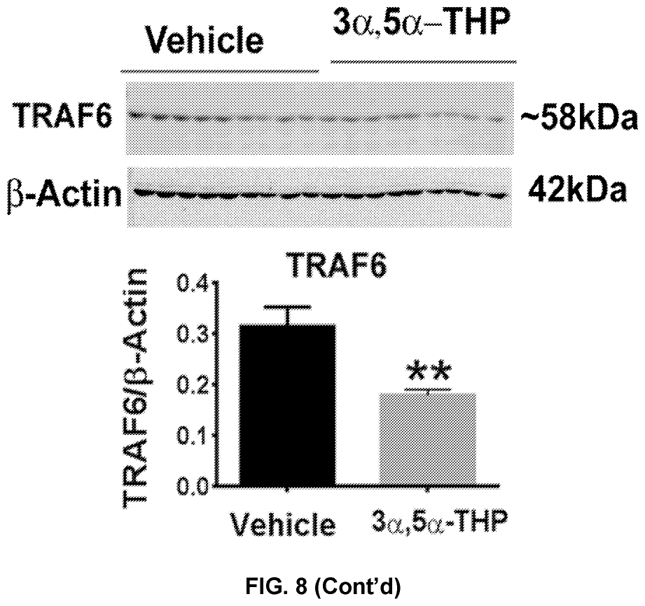

The neurosteroid 3α,5α-THP (1 μM) inhibits TLR2 and TLR7 activation and signaling in mouse macrophage ceils and brain. This extends the previously disclosed finding on inhibition of TLR4 activation and signaling. These TLRs are activated by distinct agonists but often recruited with activation of TLR4 and other inflammatory molecules. Hence the neurosteroid has greater protection against inflammatory signaling than previously disclosed. (FIGS. 7 and 8)

The neurosteroid 3α,5α-THP (1 μM) inhibits the inflammatory cytokine MCP-1 across multiple brain regions, establishing the ubiquity of this effect. Sex differences in basal MCP-1 levels are found in NAc, suggesting that endogenous levels of the steroid may impact basal levels.

The neurosteroid 3α,5α-THP (1 μM) inhibits the inflammatory cytokine IRF7. Sex difference in the inflammatory chemokine IRF7 are also found in NAc, suggesting that TLR7 activation is greater in females than males.

The neurosteroid 3α,5α-THP (1 μM) increases expression of the anti-inflammatory cytokine CX3CL1 (also known as Fracktalkine) in rat brain (NAc) and human macrophages. Anti-inflammatory cytokines are protective in many inflammatory diseases. This is another new mechanism of neurosteroid action.

Because multiple TLRs signal through MD-2, TRAF-8 and MyD-88, the specificity of the neurosteroid 3α,5α-THP on TLR2, TLRS and TLR7 signal activation was examined in RAW264.7 ceils (FIG. 7). Pam3Cys (10 μg/ml) activated TLR2 signaling, evidenced by increases in pCREB, pERK1/2, TRAF6 and pATF-2, that were sustained for 24 hrs and inhibited by 3α,5α-THP (1 μM) (50-60% compared to vehicle). Likewise, TLR7 was activated by exposure to imiquimod (1 μg/ml) for 24 hours, resulting in the 30% increase in pIRF7 and this signal was completely inhibited by 3α,5α-THP (1 μM).

In contrast, exposure to the TLRS agonist Poly-IC (25 μg/ml; 24 hrs.) resulted in a 90% increase in IP-10 (also known as CXCL10) expression, that was not altered by 3α,5α-THP (1 μM). The data suggest that 3α,5α-THP selectively inhibits the activation of TLR2, TLR4 and TLR7, all of which signal primarily through MyD88, without affecting the activation of TLR3, which primarily signals through TRIP. Coupled with recent observation (Balan et al, (2019) Sci Rep. 9(1): 1220) that 3α,5α-THP (1 μM) inhibits TLR4 signaling via blockade of TLR4 interaction with MD2, MyD88 or the GABA_{A}R α2 subunit to inhibit MyD88-dependent signaling in both RAW264.7 cells and rat brain, the data suggest that the neurosteroids selectively inhibit MyD88-dependent signaling through multiple TLRs to reduce inflammatory signaling throughout the innate immune system.

To determine if 3α,5α-THP altered TLRS or TLR7 signaling in the rat brain (FIG. 8), the P rat was again utilized, because it exhibits innate activation of TRAF6 and MCP-1, markers of TLR-MyD88-dependent signal activation in several brain regions including the ventral tegmental area (VTA), the nucleus accumbens (NAc) and the central nucleus of the amygdala (CeA) (Liu J, et al. (2011). Proc Natl Acad Sci USA 108(11): 4465-4470); June H L, et al. (2015). Neuropsychopharmacology 40(8): 1549-1559; Aurelian L, et al. (2016). Transl Psychiatry 6: e815). Systemic administration of 3α,5α-THP (15 mg/kg, I.P.) to naïve female P rats inhibited the expression of TLR7 (40%), pIRF7 (40%), and TRAF6 (40%) in P rat NAc, with no effect on IRF3. Similar results were obtained in a separate study of the male P rats. These results suggest that 3α,5α-THP inhibits TLR7 expression and activation in rat brain, consistent with the data in RAW264.7 cells suggesting that 3α,5α-THP inhibits MyD88-dependent signaling, but not TRIF-dependent signaling.

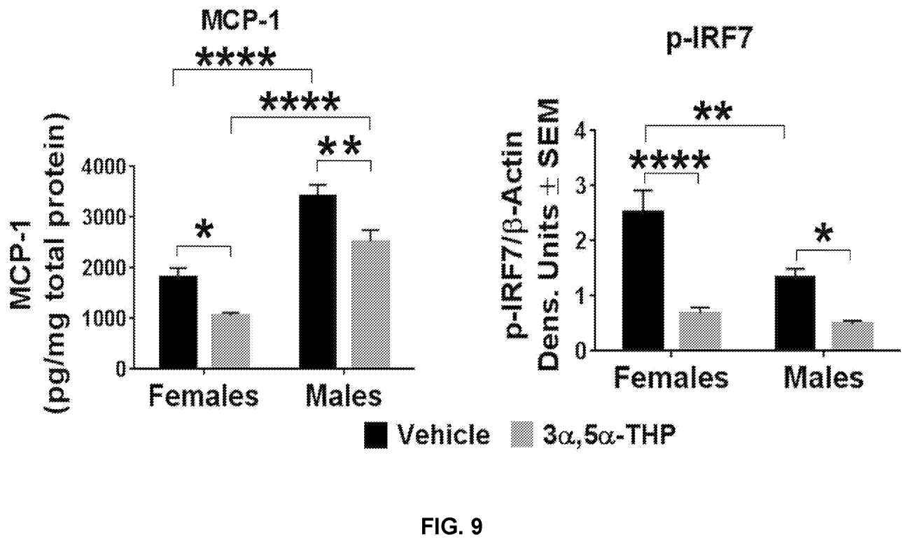

Next, potential sex differences were directly examined in baseline or 3α,5α-THP inhibition of MCP-1 and pIRF7 expression in female vs. male P rat NAc (FIG. 9). An unexpected sex difference was found in baseline MCP-1 and p-IRF7 expression, where male rats exhibited 55% higher MCP-1 protein levels compared to females, while females exhibited 45% higher p-IRF7 protein levels compared to males. Systemic administration of 3α,5α-THP (15 mg/kg, I.P.) to naïve female and male P rats inhibited the expression of MCP-1 (40% in female rat NAc; 25% in male rat NAc). Likewise, 3α,5α-THP administration to naïve female and male P rats inhibited the expression of pIRF7 to the same extent, (55% in female rat NAc; 55% in male rat NAc). There was also no sex difference in 3α,5α-THP inhibition of TRAF6 in female (40%) vs. male (45%) P rats.

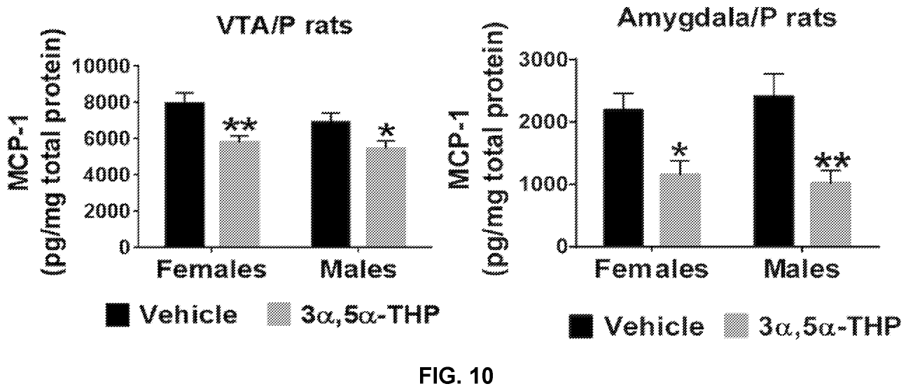

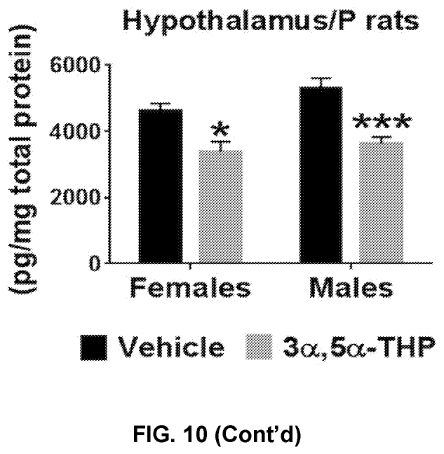

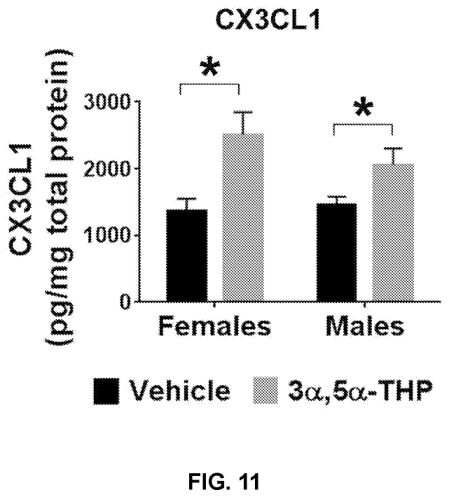

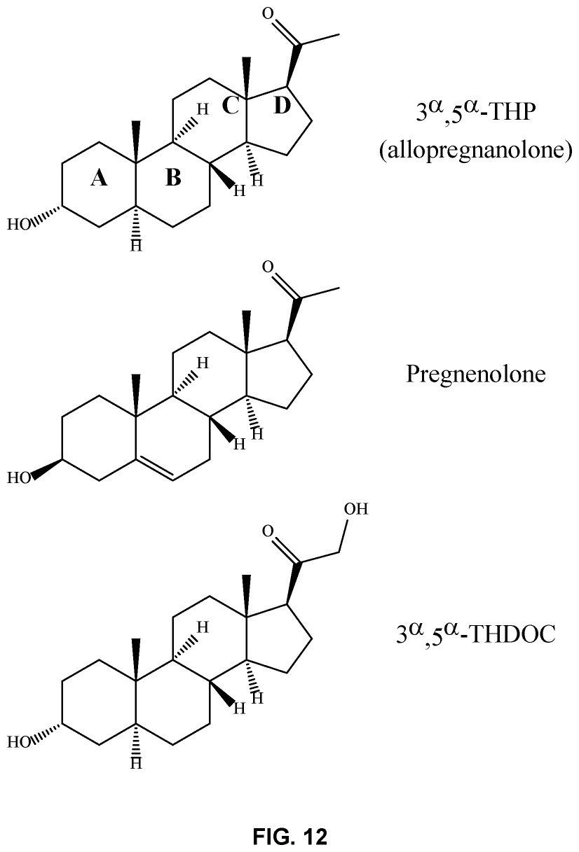

To determine if the effects of 3α,5α-THP on MCP-1 were selective for P rat NAc, the effects of 3α,5α-THP administration in VTA, Amygdala and Hypothalamus of both female and male P rats was examined. FIG. 10 indicates that 3α,5α-THP reduces MCP-1 expression in all brain areas tested, although the greater inhibition was observed in amygdala, similar to NAc.

Activation of TLR4 signaling can result in the production of both pro-inflammatory and anti-inflammatory cytokines in P rat brain, but the factors that determine the outcome of TLR4 activation are unknown. Therefore, the effects of 3α,5α-THP on the anti-inflammatory chemokine CX3CL1 (also known as Fractalkine) was examined in the P rat brain that exhibits innately activated TLR4 signaling (FIG. 11). 3α,5α-THP administration to naïve female and male P rats enhanced the expression of CX3CL1 by 90% in female rat NAc and 34% in male rat NAc. No sex difference in the effect of 3α,5α-THP was observed.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating a TLR-mediated inflammatory condition in a subject, comprising administering to the subject a neurosteroid, wherein the inflammatory condition is either not a neuropsychiatric disorder, or is a neuropsychiatric disorder that is non-responsive to GABAergic drugs.

2. The method of claim 1, wherein the neurosteroid is pregnenolone, (3α,5α)3-hydroxypregnan-20-one (3α,5α-THP), or a combination thereof.

3. The method of claim 1, wherein the neurosteroid is an inhibitor of toll-like receptor signaling or corticotropin (CRF) releasing hormone signaling.

4. The method of claim 3, wherein the neurosteroid is an inhibitor of TLR2, TLR4 or TLR7 receptor signaling.

5. The method of claim 1, wherein the TLR-mediated inflammatory condition is a neuropsychiatric disorder that is non-responsive to GABAergic drugs.

6. The method of claim 1, wherein the TLR-mediated inflammatory condition is selected from the group consisting of sepsis, gastrointestinal disease, chronic obstructive pulmonary disease (COPD), asthma, and atherosclerosis.

7. The method of claim 1, wherein the TLR-mediated inflammatory condition is selected from the group consisting of pain, stroke, seizure, alcohol detoxification, Alzheimer's disease, and dementia.

8. The method of claim 1, further comprising assaying a sample from the subject for TLR signaling in peripheral blood mononuclear cells or cerebrospinal fluid, wherein decreased TLR signaling is an indication of a therapeutically effective amount of neurosteroid.

9. The method of claim 1, further comprising increasing the amount of neurosteroid administered to the subject if decreased TLR signaling in the peripheral blood mononuclear cells or cerebrospinal fluid is not detected.

10. A method for treating a neuropsychiatric disorder in a subject in need thereof comprising
(a) detecting in a sample from the subject elevated levels of one or more of MCP-1, TNF-α, pIRF7 and HMGB1, pIRF7, and INFs; decreased levels one or more of fracktalkine and IL-10; or any combination thereof; and
(b) administering to the subject a therapeutically effective amount of a neurosteroid.

11. The method of claim 10, further comprising monitoring samples from the subject for levels of fracktalkine, IL10, MCP-1, TNF-α, pIRF7 and HMGB1, pIRF7, INFs, or any combination thereof.

12. The method of claim 10, wherein the neuropsychiatric disorder is a chronic neuropsychiatric disorder.

13. The method of claim 10, wherein the neuropsychiatric disorder is selected from a group consisting of cognitive disorders, seizure disorders, movement disorders, traumatic brain injury, secondary psychiatric disorders, substance-induced psychiatric disorders, attentional disorders, and sleep disorders.

14. The method of claim 10, wherein the neuropsychiatric disorder is alcoholism.

15. A method for identifying inhibitors of proinflammatory neuroimmune signaling comprising measuring of inhibition of MD-2 binding to TLR4 in the presence of a candidate compound, wherein the inhibition of MD-2 binding to TLR4 by a candidate compound is indicative that the candidate compound is an inhibitor of proinflammatory neuroimmune signaling.

16. The method of claim 15, wherein the candidate compound is a neurosteroid, or a modification, variant, derivative, or analog thereof.

17. The method of claim 15, wherein the inhibition of MD-2 binding to TLR4 is measured by immunoprecipitation.

18. The method of claim 15, wherein the method further comprises measuring of inhibition of upregulation of any one of, any number of, or all of, pTAK1, TRAF6, NFκB p50, phospho-NF-κB-p65, pCREB, HMGB1, MCP-1, p-IRF7, INFs and TNFα.
